(19) United States
(12) Patent Application Publication
(10) Pub. No.: US 2019/0365814 A1
(43) Pub. Date: Dec. 5, 2019

(54) VCN ENHANCER COMPOSITIONS AND METHODS OF USING THE SAME

(71) Applicant: bluebird bio, Inc., Cambridge, MA (US)

(72) Inventors: Iulia DIACONU, Cambridge, MA (US); Geoffrey B. PARSONS, Jamaica Plain, MA (US)

(73) Assignee: bluebird bio, Inc., Cambridge, MA (US)

(21) Appl. No.: 16/485,048

(22) PCT Filed: Feb. 9, 2018
(86) PCT No.: PCT/US2018/017557

§ 371 Date: Aug. 9, 2019

Related U.S. Application Data
(60) Provisional application No. 62/457,504, filed on Feb. 10, 2017.

(51) Int. Cl. A61K 35/17; A61K 47/10; C12N 15/86; A61K 9/00

(52) U.S. Cl. CPC A61K 35/17; A61K 47/10; A61K 31/4709; A61K 9/0019; C12N 15/86

(57) ABSTRACT

The invention provides improved adoptive immunotherapy compositions and methods of making the same.

### CROSS REFERENCE TO RELATED APPLICATIONS

This application claims the benefit under 35 U.S.C. § 119(e) of U.S. Provisional Application No. 62/457,504, filed Feb. 10, 2017, which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is BLBD_083_01WO_ST25.txt. The text file is 3 KB, was created on Feb. 7, 2018, and is being submitted electronically via EFS-Web, concurrent with the filing of the specification.

### BACKGROUND

### Technical Field

The present invention relates to improved T cell compositions and methods for manufacturing the same.

### Description of the Related Art

Adoptive immunotherapy is the transfer of T lymphocytes to a subject for the therapy of disease. Adoptive immunotherapy has yet unrealized potential for treating a wide variety of diseases including cancer, infectious disease, autoimmune disease, inflammatory disease, and immunodeficiency. Adoptive immunotherapy strategies that use viral vectors rely on efficient transduction to generate a clinically effective, therapeutic dose of T cells. Current technologies for generating therapeutic doses of T cells by viral transduction are limited and inefficient. In addition, existing T cell transduction methods are optimized with normal healthy donor cells and such methods do not translate well to patient cells, which have often been subjected to various cytotoxic therapies and difficult to transduce. Thus, existing T cell transduction processes produce an inferior T cell product for autologous T cell immunotherapies.

Inefficient transduction is one of the prime limiting factors preventing adoptive T cell immunotherapies from entering the clinic. Inefficient transduction also increases the expense of developing these T cell therapies because large amounts of vector are required to generate the requisite amount of transduced cells. Thus, not only would increasing the transduction efficiency of T cells provide quantum clinical benefits, but it would reduce the amount of virus required to generate the drug products and thus, reduce the costs of goods and clinical trials.

### BRIEF SUMMARY

Improved adoptive immunotherapies are contemplated herein. In particular embodiments, adoptive immunotherapies comprise genetically modified T cell compositions with increased therapeutic efficacy and methods of making and using the same. In other particular embodiments, the present invention contemplates compositions and methods for increasing transduction efficiency and VCN of human immune effector cells to yield improved adoptive immunotherapy compositions.

In various embodiments, a composition comprising a population of immune effector cells, a retroviral vector, and poloxamer 407 is contemplated.

In particular embodiments, the immune effector cells are isolated from a subject that has a cancer, an infectious disease, an autoimmune disease, an inflammatory disease, or an immunodeficiency.

In some embodiments, the immune effector cells comprise CD3+ T cells, CD4+ T cells, and/or CD8+ T cells.

In certain embodiments, the immune effector cells comprise cytotoxic T lymphocytes (CTLs), tumor infiltrating lymphocytes (TILs), or helper T cells.

In further embodiments, the immune effector cells comprise natural killer (NK) cells or natural killer T (NKT) cells.

In additional embodiments, the source of the immune effector cells is peripheral blood mononuclear cells, bone marrow, lymph nodes tissue, cord blood, thymus issue, tissue from a site of infection, ascites, pleural effusion, spleen tissue, or tumors.

In some embodiments, the retroviral vector encodes an engineered antigen receptor.

In certain embodiments, the retroviral vector encodes an engineered antigen receptor selected from the group consisting of: an engineered T cell receptor (TCR), a chimeric antigen receptor (CAR), a DARIC receptor or components thereof, and a chimeric cytokine receptor.

In further embodiments, the engineered antigen receptor comprises an extracellular binding domain that binds an antigen selected from the group consisting of: alpha folate receptor, 5T4, αvβ6 integrin, BCMA, B7-H3, B7-H6, CAIX, CD16, CD19, CD20, CD22, CD30, CD33, CD37, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, VEGFR2, and WT-1.

In further embodiments, the engineered antigen receptor comprises an extracellular binding domain that binds an antigen selected from the group consisting of: BCMA, CD19, CSPG4, PSCA, ROR1, and TAG72.

In certain embodiments, the engineered antigen receptor comprises an extracellular binding domain that binds an antigen selected from the group consisting of: BCMA, CD19, CD20, CD22, CD23, CD33, CD37, CD52, CD80, and HLA-DR.

In particular embodiments, the engineered antigen receptor comprises an extracellular binding domain that binds an antigen expressed on a cancer cell.

In particular embodiments, the engineered antigen receptor comprises an extracellular binding domain that binds an antigen expressed on a solid cancer cell.

In additional embodiments, the engineered antigen receptor comprises an extracellular binding domain that binds an antigen expressed on a liquid cancer cell.

In some embodiments, the engineered antigen receptor comprises an extracellular binding domain that binds an antigen expressed on a malignant B cell.

In further embodiments, the engineered antigen receptor comprises an extracellular binding domain that binds an antigen expressed on a malignant plasma cell.

In certain embodiments, the retroviral vector is present at an MOI of about 10 to about 30.

In some embodiments, the retroviral vector is present at an MOI of about 10 to about 25.

In particular embodiments, the retroviral vector is present at an MOI of about 10 to about 20.

In additional embodiments, the retroviral vector is present at an MOI of about 10, about 11, about 12, about 13, about 14, about 15, about 16, about 17, about 18, about 19, about 20, about 21, about 22, about 23, about 24, about 25, about 26, about 27, about 28, about 29 or about 30.

In some embodiments, the retroviral vector is a lentiviral vector.

In certain embodiments, the retroviral vector is derived from a lentivirus selected from the group consisting of: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

In further embodiments, the retroviral vector is derived from an HIV lentivirus.

In further embodiments, the retroviral vector is derived from an HIV-1 lentivirus.

In some embodiments, the population of cells comprises at least about 1×10⁶ T cells to about 1×10⁹ T cells.

In particular embodiments, the population of cells comprises at least about 1×10⁷T cells to about 1×10⁹T cells.

In additional embodiments, the population of cells comprises at least about 1×10⁸ T cells to about 1×10⁹ T cells.

In particular embodiments, the population of cells comprises at least about 1×10⁶ T cells to about 1×10⁸ T cells.

In certain embodiments, the population of cells comprises at least about 1×10⁶ T cells to about 1×10⁷ T cells.

In particular embodiments, the population of cells comprises at least about 1×10⁶ T cells, at least about 1×10⁷ T cells, at least about 1×10⁸ T cells, or at least about 1×10⁹ T cells.

In some embodiments, poloxamer 407 is present at a concentration of at least about 100 μg/mL.

In additional embodiments, poloxamer 407 is present at a concentration of at least about 200 μg/mL.

In certain embodiments, poloxamer 407 is present at a concentration of at least about 300 μg/mL.

In some embodiments, poloxamer 407 is present at a concentration of at least about 400 μg/mL.

In further embodiments, poloxamer 407 is present at a concentration of at least about 500 μg/mL.

In particular embodiments, composition further comprises a culture medium.

In some embodiments, composition further comprises a PI3K inhibitor.

In certain embodiments, composition further comprises a PI3K inhibitor selected from the group consisting of: BEZ235, LY294002, TG100713, and GDC-0941.

In further embodiments, composition further comprises a PI3K inhibitor selected from the group consisting of: BYL719, GSK2636771, TGX-221, AS25242, CAL-101, and IPI-145.

In particular embodiments, composition further comprises the PI3K inhibitor ZSTK474.

In various embodiments, a population of therapeutic immune effector cells comprising immune effector cells isolated from a subject that has a cancer, an infectious disease, an autoimmune disease, an inflammatory disease, or an immunodeficiency, wherein at least 50% of the immune effector cells are transduced and viable and wherein the immune effector cells have an average vector copy number (VCN) of about 0.5 to 5 is contemplated.

In certain embodiments, the retroviral vector transduced the immune effector cells at a multiplicity of infection (MOI) of about 10 to about 30.

In additional embodiments, the retroviral vector transduced the immune effector cells at a multiplicity of infection (MOI) of about 10 to about 25.

In particular embodiments, the retroviral vector transduced the immune effector cells at a multiplicity of infection (MOI) of about 10 to about 20.

In some embodiments, the retroviral vector transduced the immune effector cells at an MOI of about 10, about 11, about 12, about 13, about 14, about 15, about 16, about 17, about 18, about 19, about 20, about 21, about 22, about 23, about 24, about 25, about 26, about 27, about 28, about 29 or about 30.

In certain embodiments, at least 75% of the cells have been transduced.

In particular embodiments, at least 90% of the cells have been transduced.

In further embodiments, the average VCN is at least 1.0.

In additional embodiments, the average VCN is at least 1.5.

In further embodiments, the average VCN is at least 2.0.

In some embodiments, the average VCN is at least 2.5.

In certain embodiments, the average VCN is at least 3.0.

In particular embodiments, viability of the population of cells is at least 75%.

In further embodiments, viability of the population of cells is at least 85%.

In some embodiments, viability of the population of cells is at least 95%.

In particular embodiments, the immune effector cells comprise CD3+ T cells, CD4+ T cells, and/or CD8+ T cells.

In additional embodiments, the immune effector cells comprise cytotoxic T lymphocytes (CTLs), tumor infiltrating lymphocytes (TILs), or helper T cells.

In particular embodiments, the immune effector cells comprise natural killer (NK) cells or natural killer T (NKT) cells.

In certain embodiments, the source of the immune effector cells is peripheral blood mononuclear cells, bone marrow, lymph nodes tissue, cord blood, thymus issue, tissue from a site of infection, ascites, pleural effusion, spleen tissue, or tumors.

In further embodiments, the retroviral vector encodes an engineered antigen receptor.

In particular embodiments, the retroviral vector encodes an engineered antigen receptor selected from the group consisting of: an engineered T cell receptor (TCR), a chimeric antigen receptor (CAR), a DARIC receptor or components thereof, and a chimeric cytokine receptor.

In some embodiments, the engineered antigen receptor comprises an extracellular binding domain that binds an antigen selected from the group consisting of: alpha folate receptor, 5T4, αvβ6 integrin, BCMA, B7-H3, B7-H6, CAIX, CD16, CD19, CD20, CD22, CD30, CD33, CD37, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, VEGFR2, and WT-1.

In additional embodiments, the engineered antigen receptor comprises an extracellular binding domain that binds an antigen selected from the group consisting of: BCMA, CD19, CSPG4, PSCA, ROR1, and TAG72.

In certain embodiments, the engineered antigen receptor comprises an extracellular binding domain that binds an antigen selected from the group consisting of: BCMA, CD19, CD20, CD22, CD23, CD33, CD37, CD52, CD80, and HLA-DR.

In further embodiments, the engineered antigen receptor comprises an extracellular binding domain that binds an antigen expressed on a cancer cell.

In particular embodiments, the engineered antigen receptor comprises an extracellular binding domain that binds an antigen expressed on a solid cancer cell.

In additional embodiments, the engineered antigen receptor comprises an extracellular binding domain that binds an antigen expressed on a liquid cancer cell.

In some embodiments, the engineered antigen receptor comprises an extracellular binding domain that binds an antigen expressed on a malignant B cell.

In certain embodiments, the engineered antigen receptor comprises an extracellular binding domain that binds an antigen expressed on a malignant plasma cell.

In further embodiments, the retroviral vector is a lentiviral vector.

In particular embodiments, the retroviral vector is derived from a lentivirus selected from the group consisting of: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

In some embodiments, the retroviral vector is derived from an HIV lentivirus.

In certain embodiments, the retroviral vector is derived from an HIV-1 lentivirus.

In further embodiments, the population of cells comprises at least about 1×10⁶ transduced T cells to about 1×10⁹ transduced T cells.

In particular embodiments, the population of cells comprises at least about 1×10⁷ transduced T cells to about 1×10⁹ transduced T cells.

In certain embodiments, the population of cells comprises at least about 1×10⁸ transduced T cells to about 1×10⁹ transduced T cells.

In additional embodiments, the population of cells comprises at least about 1×10⁶ transduced T cells to about 1×10⁸ transduced T cells.

In further embodiments, the population of cells comprises at least about 1×10⁶ transduced T cells to about 1×10⁷ transduced T cells.

In particular embodiments, the population of cells comprises at least about 1×10⁶ transduced T cells, at least about 1×10⁷ transduced T cells, at least about 1×10⁸ transduced T cells, or at least about 1×10⁹ transduced T cells.

In various embodiments, a method of transducing a population of immune effector cells comprising culturing the cells in a culture medium, in the presence of a retrovirus, and poloxamer 407 is contemplated.

In further embodiments, the immune effector cells are isolated from a subject that has a cancer, an infectious disease, an autoimmune disease, an inflammatory disease, or an immunodeficiency.

In some embodiments, the immune effector cells comprise CD3+ T cells, CD4+ T cells, and/or CD8+ T cells.

In particular embodiments, the immune effector cells comprise cytotoxic T lymphocytes (CTLs), tumor infiltrating lymphocytes (TILs), or helper T cells.

In some embodiments, the immune effector cells comprise natural killer (NK) cells or natural killer T (NKT) cells.

In additional embodiments, the source of the immune effector cells is peripheral blood mononuclear cells, bone marrow, lymph nodes tissue, cord blood, thymus issue, tissue from a site of infection, ascites, pleural effusion, spleen tissue, or tumors.

In certain embodiments, the retroviral vector encodes an engineered antigen receptor.

In particular embodiments, the retroviral vector encodes an engineered antigen receptor selected from the group consisting of: an engineered T cell receptor (TCR), a chimeric antigen receptor (CAR), a DARIC receptor or components thereof, and a chimeric cytokine receptor.

In further embodiments, the engineered antigen receptor comprises an extracellular binding domain that binds an antigen selected from the group consisting of: alpha folate receptor, 5T4, αvβ6 integrin, BCMA, B7-H3, B7-H6, CAIX, CD16, CD19, CD20, CD22, CD30, CD33, CD37, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, VEGFR2, and WT-1.

In certain embodiments, the engineered antigen receptor comprises an extracellular binding domain that binds an antigen selected from the group consisting of: BCMA, CD19, CSPG4, PSCA, ROR1, and TAG72.

In additional embodiments, the engineered antigen receptor comprises an extracellular binding domain that binds an antigen selected from the group consisting of: BCMA, CD19, CD20, CD22, CD23, CD33, CD37, CD52, CD80, and HLA-DR.

In particular embodiments, the engineered antigen receptor comprises an extracellular binding domain that binds an antigen expressed on a cancer cell.

In further embodiments, the engineered antigen receptor comprises an extracellular binding domain that binds an antigen expressed on a solid cancer cell.

In some embodiments, the engineered antigen receptor comprises an extracellular binding domain that binds an antigen expressed on a liquid cancer cell.

In particular embodiments, the engineered antigen receptor comprises an extracellular binding domain that binds an antigen expressed on a malignant B cell.

In additional embodiments, the engineered antigen receptor comprises an extracellular binding domain that binds an antigen expressed on a malignant plasma cell.

In additional embodiments, the retroviral vector is present at an MOI of about 10 to about 30.

In further embodiments, the retroviral vector is present at an MOI of about 10 to about 25.

In further embodiments, the retroviral vector is present at an MOI of about 10 to about 20.

In particular embodiments, the retroviral vector is present at an MOI of about 10, about 11, about 12, about 13, about 14, about 15, about 16, about 17, about 18, about 19, about 20, about 21, about 22, about 23, about 24, about 25, about 26, about 27, about 28, about 29 or about 30.

In some embodiments, the retroviral vector is a lentiviral vector.

In certain embodiments, the retroviral vector is derived from a lentivirus selected from the group consisting of: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

In further embodiments, the retroviral vector is derived from an HIV lentivirus.

In additional embodiments, the retroviral vector is derived from an HIV-1 lentivirus.

In certain embodiments, the population of cells comprises at least about 1×10⁶ T cells to about 1×10⁹T cells.

In further embodiments, the population of cells comprises at least about 1×10⁷ T cells to about 1×10⁹ T cells.

In particular embodiments, the population of cells comprises at least about 1×10⁸ T cells to about 1×10⁹T cells.

In some embodiments, the population of cells comprises at least about 1×10⁶ T cells to about 1×108 T cells.

In additional embodiments, the population of cells comprises at least about 1×10⁶ T cells to about 1×10⁷ T cells.

In certain embodiments, the population of cells comprises at least about 1×10⁶ T cells, at least about 1×10⁷ T cells, at least about 1×10⁸ T cells, or at least about 1×10⁹T cells.

In particular embodiments, poloxamer 407 is present at a concentration of at least about 100 μg/mL.

In further embodiments, poloxamer 407 is present at a concentration of at least about 200 μg/mL.

In further embodiments, poloxamer 407 is present at a concentration of at least about 300 μg/mL.

In some embodiments, poloxamer 407 is present at a concentration of at least about 400 μg/mL.

In particular embodiments, poloxamer 407 is present at a concentration of at least about 500 μg/mL.

In certain embodiments, the method further comprises transducing the cells in the presence of a PI3K inhibitor.

In some embodiments, the method further comprises transducing the cells in the presence of a PI3K inhibitor selected from the group consisting of: BEZ235, LY294002, TG100713, and GDC-0941.

In additional embodiments, the method further comprises transducing the cells in the presence of a PI3K inhibitor selected from the group consisting of: BYL719, GSK2636771, TGX-221, AS25242, CAL-101, and IPI-145.

In particular embodiments, the method further comprises transducing the cells in the presence of the PI3K inhibitor ZSTK474.

In various embodiments, a method of treating a cancer in a subject in need thereof, comprising administering to the subject a therapeutically effective amount of the composition or a therapeutic population of immune effector cells contemplated herein is provided.

In additional embodiments, the cancer is selected from the group consisting of Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer.

In some embodiments, the cancer is pancreatic cancer and the extracellular binding domain binds an epitope of PSCA or MUC1

In certain embodiments, the cancer is bladder cancer and the extracellular binding domain binds an epitope of PSCA or MUC1

In further embodiments, the cancer is glioblastoma multiforme and the extracellular binding domain binds an epitope of EPHA2, EGFRvIII, or CSPG4.

In particular embodiments, the cancer is lung cancer and the extracellular binding domain binds an epitope of PSCA or GD2.

In additional embodiments, the cancer is breast cancer and the extracellular binding domain binds an epitope of CSPG4 or HER2.

In further embodiments, the cancer is melanoma and the extracellular binding domain binds an epitope of CSPG4 or GD2.

In particular embodiments, the cancer is a B-cell malignancy and the binding domain binds an epitope of BCMA.

In various embodiments, a method of treating a hematological malignancy in a subject in need thereof, comprising administering to the subject a therapeutically effective amount of the composition or a therapeutic population of immune effector cells contemplated herein is provided.

In some embodiments, the hematological malignancy is a B-cell malignancy selected from the group consisting of: multiple myeloma (MM), chronic lymphocytic leukemia (CLL), or non-Hodgkin's lymphoma (NHL).

In certain embodiments, the MM is selected from the group consisting of: overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

In particular embodiments, the NHL is selected from the group consisting of: Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma.

### BRIEF DESCRIPTION OF SEVERAL VIEWS OF THE DRAWINGS

FIG. 1 shows that F108, F127, or the combination of F108 and F127 increase VCN in PBMCs transduced with a lentiviral vector encoding a CAR compared to PBMCs transduced with the lentiviral vector encoding the CAR alone.

FIG. 2 shows the fold increase in VCN for PBMCs transduced with a lentiviral vector encoding a CAR in the presence of F108, F127, or the combination of F108 and F127 compared to PBMCs transduced with the lentiviral vector encoding the CAR alone.

FIG. 3 shows that F108 and F127 increase VCN in PBMCs transduced with a lentiviral vector encoding a transgenic TCR compared to the vector control.

FIG. 4 shows that F108 and F127 increase transgenic TCR expression in PBMCs transduced with a lentiviral vector encoding a transgenic TCR compared to the vector control.

### BRIEF DESCRIPTION OF THE SEQUENCE IDENTIFIERS

SEQ ID NOs: 1-11 set forth the amino acid sequences of various linkers.

### DETAILED DESCRIPTION

### A. Overview

Various illustrative embodiments of the invention contemplate cellular immunotherapies that comprise redirected immune effector cells with increased therapeutic efficacy and methods of making and using the same.

Adoptive immunotherapies that comprise one or more therapeutic transgenes, e.g., engineered antigen receptors, rely, in part, on sufficient expression of the therapeutic transgenes. One of the factors that influences gene expression is copy number, or how many copies of the therapeutic gene are present in the cell. Viruses, such as retrovirus, e.g., lentivirus, adenovirus, and adeno-associated virus are often used viral vectors to introduce a therapeutic transgene into the cell; a process known as transduction. Inefficient viral transduction patient derived T cells is one of the more important factors that limits the scope and applicability of redirected adoptive immunotherapies. Poor viral transduction manifests as low vector copy number (VCN) and/or a low percentage of cells transduced. Thus, a significant problem with redirected adoptive immunotherapies that use viral vectors to deliver therapeutic transgenes to T cells is inefficient viral transduction, which may give rise to a subtherapeutic drug product. Inefficient transduction also leads to higher costs of goods, e.g., it increases the cost of virus production because the more inefficient the transduction, the more virus is needed.

The adoptive immunotherapies contemplated herein, and methods of making and using the same, solve these and other problems plaguing the art.

Particular exemplary embodiments contemplate a composition of immune effector cells and poloxamer 407, optionally in combination with a PI3K inhibitor.

Certain embodiments contemplate population of immune effector cells transduced with a retroviral vector, wherein the population of cells comprises an increased number or percentage of transduced immune effector cells compared to populations of cells transduced with existing methods and compositions in the art. In another embodiment, a population of immune effector cells transduced with a retroviral vector comprises an increased VCN compared to the VCN of populations of cells transduced with existing methods and compositions in the art.

Other exemplary embodiments contemplate compositions, pharmaceutical compositions, and cell cultures comprising transduced immune effector cells.

Certain embodiments contemplate cell cultures comprising a population of immune effector cells, a retrovirus, and poloxamer 407, optionally in combination with a PI3K inhibitor.

In particular embodiments, methods for transducing immune effector cells are contemplated comprising contacting immune effector cells with a retrovirus and culturing the cell and the retrovirus in the presence of poloxamer 407, and optionally a PI3K inhibitor.

In particular embodiments, methods for treating a subject having a cancer, an infectious disease, an autoimmune disease, an inflammatory disease, or an immunodeficiency are contemplated comprising administering to the subject any of the immune effector cell-based immunotherapies contemplated herein.

Various embodiments contemplated herein will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

An “antigen (Ag)” refers to a compound, composition, or substance that can stimulate the production of antibodies or a T cell response in an animal, including compositions (such as one that includes a cancer-specific protein) that are injected or absorbed into an animal. Exemplary antigens include but are not limited to lipids, carbohydrates, polysaccharides, glycoproteins, peptides, or nucleic acids. An antigen reacts with the products of specific humoral or cellular immunity, including those induced by heterologous antigens, such as the disclosed antigens.

A “target antigen” or “target antigen of interest” is an antigen that a binding domain contemplated herein, is designed to bind. In particular embodiments, the target antigen is selected from the group consisting of: alpha folate receptor, 5T4, αvβ6 integrin, BCMA, B7-H3, B7-H6, CAIX, CD16, CD19, CD20, CD22, CD30, CD33, CD37, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, STn, TAG72, TEMs, VEGFR2, and WT-1.

In one embodiment, the antigen is an MHC-peptide complex, such as a class I MHC-peptide complex or a class II MHC-peptide complex.

As used herein, the terms, “binding domain,” “extracellular domain,” “antigen binding domain,” “extracellular binding domain,” “extracellular antigen binding domain,” “antigen-specific binding domain,” and “extracellular antigen specific binding domain,” are used interchangeably and provide a polypeptide with the ability to specifically bind to the target antigen of interest. The binding domain may be derived either from a natural, synthetic, semi-synthetic, or recombinant source.

The terms “specific binding affinity” or “specifically binds” or “specifically bound” or “specific binding” or “specifically targets” as used herein, describe binding of an antibody or antigen binding fragment thereof to a target antigen at greater binding affinity than background binding. A binding domain “specifically binds” to a target antigen, if it binds to or associates with the antigen with an affinity or Kₐ (i.e., an equilibrium association constant of a particular binding interaction with units of 1/M) of, for example, greater than or equal to about 10⁵ M⁻¹. In certain embodiments, a binding domain (or a fusion protein thereof) binds to a target with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains (or single chain fusion proteins thereof) refers to those binding domains with a Kₐ of at least 10⁷ M⁻¹, at least 10⁸ M⁻¹, at least 10⁹ M⁻¹, at least 10¹⁰M⁻¹, at least 10¹¹ M⁻¹, at least 10¹² M⁻¹, at least 10¹³ M⁻¹, or greater.

An “antibody” refers to a binding agent that is a polypeptide comprising at least a light chain or heavy chain immunoglobulin variable region which specifically recognizes and binds an epitope of an antigen, such as a lipid, carbohydrate, polysaccharide, glycoprotein, peptide, or nucleic acid containing an antigenic determinant, such as those recognized by an immune cell.

An “epitope” or “antigenic determinant” refers to the region of an antigen to which a binding agent binds.

Antibodies include antigen binding fragments thereof, such as Camel Ig, Ig NAR, Fab fragments, Fab′ fragments, F(ab)′₂ fragments, F(ab)′₃ fragments, Fv, single chain Fv proteins (“scFv”), bis-scFv, (scFv)₂, minibodies, diabodies, triabodies, tetrabodies, disulfide stabilized Fv proteins (“dsFv”), and single-domain antibody (sdAb, Nanobody) and portions of full length antibodies responsible for antigen binding. The term antibody also includes human antibodies and genetically engineered forms such as chimeric antibodies (for example, humanized murine antibodies), heteroconjugate antibodies (such as, bispecific antibodies) and antigen binding fragments thereof. See also, Pierce Catalog and Handbook, 1994-1995 (Pierce Chemical Co., Rockford, Ill.); Kuby, J., Immunology, 3_{rd} Ed., W. H. Freeman & Co., New York, 1997.

A “linker” refers to a plurality of amino acid residues between the various polypeptide domains, e.g., between V_{H} and V_{L} domains, added for appropriate spacing and conformation of the molecule. In particular embodiments, the linker is a variable region linking sequence. A “variable region linking sequence,” is an amino acid sequence that connects the V_{H} and V_{L} domains and provides a spacer function compatible with interaction of the two sub-binding domains so that the resulting polypeptide retains a specific binding affinity to the same target molecule as an antibody that comprises the same light and heavy chain variable regions. In particular embodiments, a linker separates one or more heavy or light chain variable domains, hinge domains, multimerization domains, transmembrane domains, co-stimulatory domains, and/or primary signaling domains.

Illustrated examples of linkers suitable for use in particular embodiments contemplated herein include, but are not limited to the following amino acid sequences: GGG; DGGGS (SEQ ID NO: 1); TGEKP (SEQ ID NO: 2) (see, e.g., Liu et al., PNAS 5525-5530 (1997)); GGRR (SEQ ID NO: 3) (Pomerantz et al. 1995, supra); (GGGGS)ₙ wherein n=1, 2, 3, 4 or 5 (SEQ ID NO: 4) (Kim et al., PNAS 93, 1156-1160 (1996.); EGKSSGSGSESKVD (SEQ ID NO: 5) (Chaudhary et al., 1990, Proc. Natl. Acad. Sci. U.S.A. 87:1066-1070); KESGSVSSEQLAQFRSLD (SEQ ID NO: 6) (Bird et al., 1988, Science 242:423-426), GGRRGGGS (SEQ ID NO: 7); LRQRDGERP (SEQ ID NO: 8); LRQKDGGGSERP (SEQ ID NO: 9); LRQKD(GGGS)₂ ERP (SEQ ID NO: 10). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods. In one embodiment, the linker comprises the following amino acid sequence: GSTSGSGKPGSGEGSTKG (SEQ ID NO: 11) (Cooper et al., Blood, 101(4): 1637-1644 (2003)).

A “spacer domain,” refers to a polypeptide that separates two domains. In one embodiment, a spacer domain moves an antigen binding domain away from the effector cell surface to enable proper cell/cell contact, antigen binding and activation (Patel et al., Gene Therapy, 1999; 6: 412-419). In particular embodiments, a spacer domain separates one or more heavy or light chain variable domains, multimerization domains, transmembrane domains, co-stimulatory domains, and/or primary signaling domains. The spacer domain may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region.

A “hinge domain,” refers to a polypeptide that plays a role in positioning the antigen binding domain away from the effector cell surface to enable proper cell/cell contact, antigen binding and activation. In particular embodiments, polypeptides may comprise one or more hinge domains between the binding domain and the multimerization domain, between the binding domain and the transmembrane domain (TM), or between the multimerization domain and the transmembrane domain. The hinge domain may be derived either from a natural, synthetic, semi-synthetic, or recombinant source. The hinge domain can include the amino acid sequence of a naturally occurring immunoglobulin hinge region or an altered immunoglobulin hinge region.

A “multimerization domain,” as used herein, refers to a polypeptide that preferentially interacts or associates with another different polypeptide directly or via a bridging molecule, wherein the interaction of different multimerization domains substantially contributes to or efficiently promotes multimerization (i.e., the formation of a dimer, trimer, or multipartite complex, which may be a homodimer, heterodimer, homotrimer, heterotrimer, homomultimer, heteromultimer). A multimerization domain may be derived either from a natural, synthetic, semi-synthetic, or recombinant source.

Illustrative examples of multimerization domains suitable for use in particular embodiments contemplated herein include an FKBP polypeptide, an FRB polypeptide, a calcineurin polypeptide, a cyclophilin polypeptide, a bacterial DHFR polypeptide, a PYL1 polypeptide, an ABI1 polypeptide, a GIB1 polypeptide, a GAI polypeptide, or variants thereof.

A “bridging factor” refers to a molecule that associates with and that is disposed between two or more multimerization domains. In particular embodiments, multimerization domains substantially contribute to or efficiently promote formation of a polypeptide complex only in the presence of a bridging factor. In particular embodiments, multimerization domains do not contribute to or do not efficiently promote formation of a polypeptide complex in the absence of a bridging factor. Illustrative examples of bridging factors suitable for use in particular embodiments contemplated herein include, but are not limited to rapamycin (sirolimus) or a rapalog thereof, coumermycin or a derivative thereof, gibberellin or a derivative thereof, abscisic acid (ABA) or a derivative thereof, methotrexate or a derivative thereof, cyclosporin A or a derivative thereof, FKCsA or a derivative thereof, trimethoprim (Tmp)-synthetic ligand for FKBP (SLF) or a derivative thereof, or any combination thereof.

Rapamycin analogs (rapalogs) include, but are not limited to those disclosed in U.S. Pat. No. 6,649,595, which rapalog structures are incorporated herein by reference in their entirety. In certain embodiments, a bridging factor is a rapalog with substantially reduced immunosuppressive effect as compared to rapamycin. In a preferred embodiment, the rapalog is AP21967 derivatives (also known as C-16-(S)-7-methylindolerapamycin, IC₅₀=10 nM, a chemically modified non-immunosuppressive rapamycin analogue).

As used herein, “anchor domain” refers to an amino acid sequence or other molecule that promotes tethering, anchoring or association of a dimerizable salvage receptor to a cell surface. Exemplary anchor domains include an amino acid sequence with a structure that is stable in a cell membrane or an amino acid sequence that promotes the addition of a glycolipid (also known as glycosyl phosphatidylinositols or GPIs), or the like. By way of background, a GPI molecule is post-translationally attached to a protein target by a transamidation reaction, which results in the cleavage of a carboxy-terminal GPI signal sequence (see, e.g., White et al., J. Cell Sci. 113:721, 2000) and the simultaneous transfer of the already synthesized GPI anchor molecule to the newly formed carboxy-terminal amino acid (see www.ncbi.nlm.nih.gov/books/NBK20711 for exemplary GPI anchors, which GPI anchors are incorporated by reference in their entirety). In certain embodiments, an anchor domain is a hydrophobic domain (e.g., transmembrane domain) or a GPI signal sequence. In some embodiments, a nucleic acid molecule encoding a polypeptide contemplated herein comprises an anchor domain, optionally wherein the anchor domain is a GPI molecule.

A “transmembrane domain” is a domain that anchors a polypeptide to the plasma membrane of a cell. The TM domain may be derived either from a natural, synthetic, semi-synthetic, or recombinant source.

An “intracellular signaling domain,” refers to a polypeptide that participates in transducing the message of effective binding of a target antigen by a receptor expressed on an immune effector cell to into the interior of the immune effector cell to elicit effector cell function, e.g., activation, cytokine production, proliferation and cytotoxic activity, including the release of cytotoxic factors, or other cellular responses elicited with antigen binding to the receptor expressed on the immune effector cell.

The term “effector function” refers to a specialized function of an immune effector cell. Effector function of the T cell, for example, may be cytolytic activity or help or activity including the secretion of a cytokine. Thus, the term “intracellular signaling domain” refers to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal.

It is known that signals generated through the TCR alone are insufficient for full activation of the T cell and that a secondary or co-stimulatory signal is also required. Thus, T cell activation can be said to be mediated by two distinct classes of intracellular signaling domains: primary signaling domains that initiate antigen-dependent primary activation through the TCR (e.g., a TCR/CD3 complex) and co-stimulatory signaling domains that act in an antigen-independent manner to provide a secondary or co-stimulatory signal.

A “primary signaling domain” refers to a signaling domain that regulates the primary activation of the TCR complex either in a stimulatory way, or in an inhibitory way. Primary signaling domains that act in a stimulatory manner may contain signaling motifs which are known as immunoreceptor tyrosine-based activation motifs or ITAMs. Illustrative examples of ITAM containing primary signaling domains that are suitable for use in particular embodiments include, but are not limited to those derived from FcRγ, FcRβ, CD3γ, CD3δ, CD3ε, CD3ζ, CD22, CD79a, CD79b, and CD66d.

As used herein, the term, “co-stimulatory signaling domain,” or “co-stimulatory domain”, refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules from which co-stimulatory domains may be isolated include, but are not limited to: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70.

As used herein, the term “cancer” relates generally to a class of diseases or conditions in which abnormal cells divide without control and can invade nearby tissues.

As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor.

As used herein, the term “benign” or “non-malignant” refers to tumors that may grow larger but do not spread to other parts of the body. Benign tumors are self-limited and typically do not invade or metastasize.

A “cancer cell” refers to an individual cell of a cancerous growth or tissue. Cancer cells include both solid cancers and liquid cancers. A “tumor” or “tumor cell” refers generally to a swelling or lesion formed by an abnormal growth of cells, which may be benign, pre-malignant, or malignant. Most cancers form tumors, but liquid cancers, e.g., leukemia, do not necessarily form tumors. For those cancers that form tumors, the terms cancer (cell) and tumor (cell) are used interchangeably. The amount of a tumor in an individual is the “tumor burden” which can be measured as the number, volume, or weight of the tumor.

The term “relapse” refers to the diagnosis of return, or signs and symptoms of return, of a cancer after a period of improvement or remission.

“Remission,” is also referred to as “clinical remission,” and includes both partial and complete remission. In partial remission, some, but not all, signs and symptoms of cancer have disappeared. In complete remission, all signs and symptoms of cancer have disappeared, although cancer still may be in the body.

“Refractory” refers to a cancer that is resistant to, or non-responsive to, therapy with a particular therapeutic agent. A cancer can be refractory from the onset of treatment (i.e., non-responsive to initial exposure to the therapeutic agent), or as a result of developing resistance to the therapeutic agent, either over the course of a first treatment period or during a subsequent treatment period.

“Antigen negative” refers to a cell that does not express antigen or expresses a neglible amount of antigen that is undetectable. In one embodiment, antigen negative cells do not bind receptors directed to the antigen. In one embodiment, antigen negative cells do not substantially bind receptors directed to the antigen.

As used herein, the term “patient” refers to a subject that has been diagnosed with a particular disease, disorder, or condition that can be treated with the compositions and methods contemplated herein.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction of the disease or condition, or the delaying of the progression of the disease or condition, e.g., delaying tumor outgrowth. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, the phrase “ameliorating at least one symptom of” refers to decreasing one or more symptoms of the disease or condition for which the subject is being treated. In particular embodiments, the disease or condition being treated is a cancer, wherein the one or more symptoms ameliorated include, but are not limited to, weakness, fatigue, shortness of breath, easy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function).

By “enhance” or “promote,” or “increase” or “expand” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, persistence, and/or an increase in cancer cell killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

By “maintain,” or “preserve,” or “maintenance,” or “no change,” or “no substantial change,” or “no substantial decrease” refers generally to the ability of a composition contemplated herein to produce, elicit, or cause a substantially similar or comparable physiological response (i.e., downstream effects) in a cell, as compared to the response caused by either vehicle, a control molecule/composition, or the response in a particular cell lineage. A comparable response is one that is not significantly different or measurable different from the reference response.

“Transfection” refer to the process of introducing naked DNA into cells by non-viral methods.

“Infection” refers to the process of introducing foreign DNA into cells using a viral vector.

“Transduction” refers to the introduction of foreign DNA into a cell's genome using a viral vector.

“Vector copy number” or “VCN” refers to the number of copies of a vector, or portion thereof, in a cell's genome. The average VCN may be determined from a population of cells or from individual cell colonies. Exemplary methods for determining VCN include polymerase chain reaction (PCR) and flow cytometry.

“Transduction efficiency” refers to the percentage of cells transduced with at least one copy of a vector. For example if 1×10⁶ cells are exposed to a virus and 0.5×10⁶ cells are determined to have a least one copy of a virus in their genome, then the transduction efficiency is 50%. Exemplary methods for determining transduction efficiency include PCR and flow cytometry. In various embodiments, the phrases “number of lentiviral vector positive cells” or “percent lentiviral vector positive cells” is used indicate the transduction efficiency.

As used herein, the term “retrovirus” refers an RNA virus that reverse transcribes its genomic RNA into a linear double-stranded DNA copy and subsequently covalently integrates its genomic DNA into a host genome. Retroviruses are a common tool for gene delivery (Miller, 2000, Nature. 357: 455-460). Once the virus is integrated into the host genome, it is referred to as a “provirus.” The provirus serves as a template for RNA polymerase II and directs the expression of RNA molecules encoded by the virus.

Illustrative retroviruses include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

Retroviral vectors, and more particularly, lentiviral vectors, may be used in practicing the present invention. Accordingly, the term “retrovirus” or “retroviral vector,” as used herein is meant to include “lentivirus” and “lentiviral vectors” respectively.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a retroviral vector or plasmid containing structural and functional genetic elements, or portions thereof, including LTRs that are primarily derived from a lentivirus. The term “hybrid” refers to a vector, LTR or other nucleic acid containing both retroviral, e.g., lentiviral, sequences and non-lentiviral viral sequences. In one embodiment, a hybrid vector refers to a vector or transfer plasmid comprising retroviral, e.g., lentiviral, sequences for reverse transcription, replication, integration and/or packaging.

In particular embodiments, the terms “lentiviral vector” and “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles of the invention and are present in DNA form in the DNA plasmids of the invention.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, R and U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

As used herein, the term “packaging signal” or “packaging sequence” refers to sequences located within the retroviral genome which are required for insertion of the viral RNA into the viral capsid or particle, see e.g., Clever et al., 1995. J. of Virology, Vol. 69, No. 4; pp. 2101-2109. Several retroviral vectors use the minimal packaging signal (also referred to as the psi [Ψ] or [Ψ+] sequence) needed for encapsidation of the viral genome. Thus, as used herein, the terms “packaging sequence,” “packaging signal,” “psi” and the symbol “Ψ,” are used in reference to the non-coding sequence required for encapsidation of retroviral RNA strands during viral particle formation.

In various embodiments, vectors comprise modified 5′ LTR and/or 3′ LTRs. Modifications of the 3′ LTR are often made to improve the safety of lentiviral or retroviral systems by rendering viruses replication-defective. The skilled artisan would be able to determine whether an LTR is modified by comparison to a reference LTR. As used herein, the term “replication-defective” refers to virus that is not capable of complete, effective replication such that infective virions are not produced (e.g., replication-defective lentiviral progeny). The term “replication-competent” refers to wild-type virus or mutant virus that is capable of replication, such that viral replication of the virus is capable of producing infective virions (e.g., replication-competent lentiviral progeny).

“Self-inactivating” (SIN) vectors refers to replication-defective vectors, e.g., retroviral or lentiviral vectors, in which the right (3′) LTR enhancer-promoter region, known as the U3 region, has been modified (e.g., by deletion and/or substitution) to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In a further embodiment of the invention, the 3′ LTR is modified such that the U5 region is replaced, for example, with a heterologous or synthetic poly(A) sequence, one or more insulator elements, and/or an inducible promoter. It should be noted that modifications to the LTRs such as modifications to the 3′ LTR, the 5′ LTR, or both 3′ and 5′ LTRs, are also included in the invention.

An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters.

As used herein, the term “FLAP element” refers to a nucleic acid whose sequence includes the central polypurine tract and central termination sequences (cPPT and CTS) of a retrovirus, e.g., HIV-1 or HIV-2. In some embodiments, the terms “FLAP element” and “cPPT/FLAP” are used interchangeably to refer to the foregoing FLAP element. Suitable FLAP elements are described in U.S. Pat. No. 6,682,907 and in Zennou, et al., 2000, Cell, 101:173. During HIV-1 reverse transcription, central initiation of the plus-strand DNA at the central polypurine tract (cPPT) and central termination at the central termination sequence (CTS) lead to the formation of a three-stranded DNA structure: the HIV-1 central DNA flap. While not wishing to be bound by any theory, the DNA flap may act as a cis-active determinant of lentiviral genome nuclear import and/or may increase the titer of the virus. In particular embodiments, the retroviral or lentiviral vector backbones comprise one or more FLAP elements upstream or downstream of the heterologous genes of interest in the vectors. For example, in particular embodiments a vector includes a FLAP element. In one embodiment, a vector of the invention comprises a FLAP element isolated from HIV-1.

In one embodiment, retroviral or lentiviral transfer vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE). Generally, the RNA export element is placed within the 3′ UTR of a gene, and can be inserted as one or multiple copies.

In particular embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal polyA sequence (e.g., AATAAA, ATTAAA, AGTAAA). In particular embodiments the poly(A) sequence is an SV40 polyA sequence, a bovine growth hormone polyA sequence (BGHpA), a rabbit β-globin polyA sequence (rβgpA), or another suitable heterologous or endogenous polyA sequence known in the art.

As used herein, the term “agent” encompasses poloxamers, small organic molecules, and PI3K inhibitors, including without limitation, all analogs and derivatives thereof.

A “small molecule,” “small organic molecule,” or “small molecule compound” refers to a low molecular weight compound that has a molecular weight of less than about 5 kD, less than about 4 kD, less than about 3 kD, less than about 2 kD, less than about 1 kD, or less than about 0.5 kD. In particular embodiments, small molecules can include, nucleic acids, peptides, peptidomimetics, peptoids, other small organic compounds or drugs, and the like. Libraries of chemical and/or biological mixtures, such as fungal, bacterial, or algal extracts, are known in the art and can be screened with any of the assays of the invention. Examples of methods for the synthesis of molecular libraries can be found in: (Carell et al., 1994a; Carell et al., 1994b; Cho et al., 1993; DeWitt et al., 1993; Gallop et al., 1994; Zuckermann et al., 1994).

In reference to agents, such as organic molecules, “analog” or “derivative” relates to a molecule that is similar to another chemical substance in structure and function, often differing structurally by a single element or group, but may differ by differ by modification of more than one group (e.g., 2, 3, or 4 groups) if it retains the same function as the parental chemical. Such modifications are routine to persons skilled in the art, and include, for example, additional or substituted chemical moieties, such as esters or amides of an acid, protecting groups such as a benzyl group for an alcohol or thiol, and tert-butoxylcarbonyl groups for an amine. Also included are modifications to alkyl side chains, such as alkyl substitutions (e.g., methyl, dimethyl, ethyl, etc.), modifications to the level of saturation or unsaturation of side chains, and the addition of modified groups such as substituted phenyl and phenoxy. Derivatives may also include conjugates, such as biotin or avidin moieties, enzymes such as horseradish peroxidase and the like, and including radio-labeled, bioluminescent, chemoluminescent, or fluorescent moieties. Also, moieties may be added to the agents described herein to alter their pharmacokinetic properties, such as to increase half-life in vivo or ex vivo, or to increase their cell penetration properties, among other desirable properties. Also included are prodrugs, which are known to enhance numerous desirable qualities of pharmaceuticals (e.g., solubility, bioavailability, manufacturing, etc.) (see, e.g., WO/2006/047476 for exemplary EP agonist prodrugs, which is incorporated by reference for its disclosure of such agonists).

As used herein, the terms “polynucleotide” or “nucleic acid” refers to genomic DNA (gDNA), complementary DNA (cDNA) or DNA. Polynucleotides include single and double stranded polynucleotides, either recombinant, synthetic, or isolated. In some embodiments, polynucleotide refers to messenger RNA (mRNA), RNA, genomic RNA (gRNA), plus strand RNA (RNA(+)), minus strand RNA (RNA(−)). As used here, the terms “polyribonucleotide” or “ribonucleic acid” also refer to messenger RNA (mRNA), RNA, genomic RNA (gRNA), plus strand RNA (RNA(+)), minus strand RNA (RNA(−)) and inhibitory RNAs, including but not limited to siRNA, shRNA, piRNA, miRNA or microRNA, and shRNAs embedded in a microRNA backbone (shmir). Preferably, polynucleotides of the invention include polynucleotides or variants having at least about 50%, 55%, 60%, 65%, 70%, 75%, 80%, 85%, 90%, 95%, 96%, 97%, 98%, 99% or 100% sequence identity to any of the reference sequences described herein (see, e.g., Sequence Listing), typically where the variant maintains at least one biological activity of the reference sequence. In various illustrative embodiments, viral vector and transfer plasmid polynucleotide sequences and compositions comprising the same are contemplated. In particular embodiments, polynucleotides encoding a therapeutic polypeptide including, but not limited to, engineered antigen receptors.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted, or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

As used herein, the term “isolated” means material, e.g., a polynucleotide, a polypeptide, a cell, that is substantially or essentially free from components that normally accompany it in its native state. In particular embodiments, the term “obtained” or “derived” is used synonymously with isolated. For example, an “isolated polynucleotide,” as used herein, refers to a polynucleotide that has been purified from the sequences which flank it in a naturally-occurring state, e.g., a DNA fragment that has been removed from the sequences that are normally adjacent to the fragment.

The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. Vectors may comprise one, two, three, four, five or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In a preferred embodiment, the nucleic acid cassette contains the sequence of a therapeutic gene used to treat, prevent, or ameliorate a genetic disorder. The cassette can be removed and inserted into a plasmid or viral vector as a single unit.

As used herein, the term “polynucleotide(s)-of-interest” refers to one or more polynucleotides, e.g., a polynucleotide encoding a polypeptide (i.e., a polypeptide-of-interest), inserted into an expression vector that is desired to be expressed. In preferred embodiments, vectors and/or plasmids comprise one or more polynucleotides-of-interest, e.g., an engineered antigen receptor.

Polynucleotides, regardless of the length of the coding sequence itself, may be combined with other DNA sequences, such as promoters and/or enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

The term “expression control sequence” refers to a polynucleotide sequence that comprises one or more promoters, enhancers, or other transcriptional control elements or combinations thereof that are capable of directing, increasing, regulating, or controlling the transcription or expression of an operatively linked polynucleotide. In particular embodiments, vectors of the invention comprise one or more expression control sequences that are specific to particular cells, cell types, or cell lineages e.g., target cells; that is, expression of polynucleotides operatively linked to an expression control sequence specific to particular cells, cell types, or cell lineages is expressed in target cells and not in other non-target cells. Each one of the one or more expression control sequences in a vector that are cell specific may express in the same or different cell types depending on the therapy desired. In preferred embodiments, vectors comprise one or more expression control sequences specific to immune effector cells or T cells.

The term “promoter” as used herein refers to a recognition site of a polynucleotide (DNA or RNA) to which an RNA polymerase binds. The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

In particular embodiments, a vector comprises exogenous, endogenous, or heterologous control sequences such as promoters and/or enhancers. An “endogenous” control sequence is one which is naturally linked to a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated. A “synthetic” control sequence may comprise elements of one more endogenous and/or exogenous sequences, and/or sequences determined in vitro or in silico that provide optimal promoter and/or enhancer activity for the particular gene therapy.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer or other expression control sequence) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively. Illustrative ubiquitous expression control sequences include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., (2007) Nature Biotechnology 25, 1477-1482), a Ubiquitin C promoter (UBC), a phosphoglycerate kinase-1 (PGK) promoter, a cytomegalovirus enhancer/chicken β-actin (CAG) promoter, and a β-actin promoter.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments of the invention provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., (2003) Gene, 323:67), the cumate inducible gene switch (WO 2002/088346), tetracycline-dependent regulatory systems, etc.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., (1990) Trends Biochem Sci 15(12):477-83) and Jackson and Kaminski. (1995) RNA 1(10):985-1000. In particular embodiments, a vector includes one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides.

As used herein, the term “Kozak sequence” refers to a short nucleotide sequence that greatly facilitates the initial binding of mRNA to the small subunit of the ribosome and increases translation. The consensus Kozak sequence is (GCC)RCCATGG, where R is a purine (A or G) (Kozak, (1986) Cell. 44(2):283-92, and Kozak, (1987) Nucleic Acids Res. 15(20):8125-48). In particular embodiments, the vectors contemplated by the invention, comprise polynucleotides that have a consensus Kozak sequence and that encode a desired polypeptide.

In various embodiments, vectors are used to increase, establish and/or maintain the expression of one or more polypeptides. The terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. Illustrative examples of polypeptides include, but are not limited to engineered antigen receptors.

Particular embodiments contemplated herein, also include polypeptide “variants.” The recitation polypeptide “variant” refers to polypeptides that are distinguished from a reference polypeptide by the addition, deletion, truncations, modifications, and/or substitution of at least one amino acid residue, and that retain a biological activity. In certain embodiments, a polypeptide variant is distinguished from a reference polypeptide by one or more substitutions, which may be conservative or non-conservative, as known in the art.

In certain embodiments, a variant polypeptide includes an amino acid sequence having at least about 50%, 55%, 60%, 65%, 70%, 75%, 80%, 85%, 90%, 91%, 92%, 93%, 94%, 95%, 96%, 97%, 98%, 99% or more sequence identity or similarity to a corresponding sequence of a reference polypeptide. In certain embodiments, amino acid additions or deletions occur at the C-terminal end and/or the N-terminal end of the reference polypeptide.

As noted above, polypeptides of the invention may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985) Proc. Natl. Acad. Sci. USA. 82: 488-492, Kunkel et al., (1987) Methods in Enzymol, 154: 367-382, U.S. Pat. No. 4,873,192, Watson, J. D. et al., (1987) Molecular Biology of the Gene, Fourth Edition, Benjamin/Cummings, Menlo Park, Calif., and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4⁺ presenting cells. In a preferred embodiment, lentiviral envelope proteins are pseudotyped with VSV-G. In one embodiment, packaging cells produce recombinant retrovirus, e.g., lentivirus, pseudotyped with the VSV-G envelope glycoprotein.

In the following description, certain specific details are set forth in order to provide a thorough understanding of various illustrative embodiments of the invention contemplated herein. However, one skilled in the art will understand that particular illustrative embodiments may be practiced without these details. In addition, it should be understood that the individual vectors, or groups of vectors, derived from the various combinations of the structures and substituents described herein, are disclosed by the present application to the same extent as if each vector or group of vectors was set forth individually. Thus, selection of particular vector structures or particular substituents is within the scope of the present disclosure.

### C. Agents

Various embodiments contemplated herein, arise from the unexpected finding that transduction efficiency and/or VCN is significantly increased by contacting immune effector cells, in vitro, ex vivo, or in vivo, with a retrovirus and one or more agents that increase transduction efficiency or VCN contemplated herein. In various embodiments, transduction efficiency is significantly increased by contacting cells, in vitro, ex vivo, or in vivo, with a retrovirus, and poloxamer 407. In various embodiments, transduction efficiency is significantly increased by contacting cells, in vitro, ex vivo, or in vivo, with a retrovirus, and a poloxamer, e.g., poloxamer 407, optionally in combination a PI3K inhibitor or inhibitor of the PI3K pathway.

It was surprising that poloxamer 407 increases the transduction of immune effector cells with retrovirus in view of U.S. Patent Application Publication No. 20150064788, which teaches away from using poloxamer 407 for this purpose.

1. Poloxamer 407

In various embodiments, transduction efficiency is increased in a population of immune effector cells comprising culturing the cells in the presence of a retrovirus and a poloxamer, e.g., poloxamer 407.

In particular embodiments, transduction efficiency is increased in a population of immune effector cells comprising culturing the cells in the presence of a retrovirus and poloxamer 407 and optionally a PI3K inhibitor.

In certain embodiments, transduction efficiency is increased in a population of immune effector cells comprising culturing the cells in the presence of a retrovirus, poloxamer 407 and ZSTK474.

“Poloxamer” refers to a non-ionic triblock copolymer composed of a central hydrophobic chain of polyoxypropylene flanked by two hydrophilic chains of polyoxyethylene. Poloxamers are also known by the trade name of “Pluronics” or “Synperonics” (BASF). The block copolymer can be represented by the following formula: HO(C₂H₄0)ₓ(C₃H₆0)_{y}(C₂H₄0)_{z}H.

The synthesis of block copolymers yields a population of polymers that have an average molecular weight. Thus, in particular embodiments, the term “poloxamer” as used herein can be used interchangeably with the term “poloxamers” (representing an entity of several poloxamers, also referred to as mixture of poloxamers) if not explicitly stated otherwise. The term “average” in relation to the number of monomer units or molecular weight of (a) poloxamer(s) as used herein is a consequence of the technical inability to produce poloxamers all having the identical composition and thus the identical molecular weight. Poloxamers produced according to state of the art methods will be present as a mixture of poloxamers each showing a variability as regards their molecular weight, but the mixture as a whole averaging the molecular weight specified herein. BASF and Sigma Aldrich are suitable sources of poloxamers for use in particular embodiments contemplated herein.

The inventors have surprisingly found that poloxamer 407 as defined herein alone or in combination with a PI3K inhibitor significantly enhances the transduction efficiency and/or VCN of retroviral vectors in adherent and suspension target cells without essentially affecting their viability.

In one embodiment, a poloxamer suitable for use in particular embodiments contemplated herein is selected from the group consisting of: poloxamer 288, poloxamer 335, poloxamer 338, and poloxamer 407.

In a preferred embodiment, the poloxamer is poloxamer 407.

Poloxamer 407 (F127; HO(C₂H₄0)ₓ(C₃H₆0)_{y}(C₂H₄0)_{z}H; x+y=200.45, z=65.17; average molecular weight of 12.6 kDa) is used to increase transduction efficiency and/or VCN in a population of immune effector cells comprising T cells. F127 can be used alone, or in combination with a PI3K inhibitor to increase transduction efficiency and/or VCN.

In a particular embodiment, a population of T cells is cultured in the presence of a retrovirus and poloxamer 407 to increase transduction efficiency and/or VCN.

2. PI3K Inhibitors

In various embodiments, transduction efficiency is increased in a population of immune effector cells comprising T cells and culturing the cells in the presence of a retrovirus and a poloxamer and a PI3K inhibitor.

As used herein, the term “PI3K inhibitor” refers to a nucleic acid, peptide, compound, or small organic molecule that binds to and inhibits at least one activity of PI3K. The PI3K proteins can be divided into three classes, class 1 PI3Ks, class 2 PI3Ks, and class 3 PI3Ks. Class 1 PI3Ks exist as heterodimers consisting of one of four p110 catalytic subunits (p110α, p110β, p110δ, and p110γ) and one of two families of regulatory subunits. A PI3K inhibitor of the present invention preferably targets the class 1 PI3K inhibitors. In one embodiment, a PI3K inhibitor will display selectivity for one or more isoforms of the class 1 PI3K inhibitors (i.e., selectivity for p110α, p110β, p110δ, and p110γ or one or more of p110α, p110β, p110δ, and p110γ). In another aspect, a PI3K inhibitor will not display isoform selectivity and be considered a “pan-PI3K inhibitor.” In one embodiment, a PI3K inhibitor will compete for binding with ATP to the PI3K catalytic domain.

In certain embodiments, a PI3K inhibitor can, for example, target PI3K as well as additional proteins in the PI3K-AKT-mTOR pathway. In particular embodiments, a PI3K inhibitor that targets both mTOR and PI3K can be referred to as either a mTOR inhibitor or a PI3K inhibitor. A PI3K inhibitor that only targets PI3K can be referred to as a selective PI3K inhibitor. In one embodiment, a selective PI3K inhibitor can be understood to refer to an agent that exhibits a 50% inhibitory concentration with respect to PI3K that is at least 10-fold, at least 20-fold, at least 30-fold, at least 50-fold, at least 100-fold, at least 1000-fold, or more, lower than the inhibitor's IC50 with respect to mTOR and/or other proteins in the pathway.

In a particular embodiment, exemplary PI3K inhibitors inhibit PI3K with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 μM, 50 μM, 25 μM, 10 μM, 1 μM, or less. In one embodiment, a PI3K inhibitor inhibits PI3K with an IC50 from about 2 nM to about 100 nm, more preferably from about 2 nM to about 50 nM, even more preferably from about 2 nM to about 15 nM.

Illustrative examples of PI3K inhibitors suitable for use increasing transduction of immune effector cells include, but are not limited to, BKM120 (class 1 PI3K inhibitor, Novartis), XL147 (class 1 PI3K inhibitor, Exelixis), (pan-PI3K inhibitor, GlaxoSmithKline), and PX-866 (class 1 PI3K inhibitor; p110α, p110β, and p110γ isoforms, Oncothyreon).

Other illustrative examples of selective PI3K inhibitors include, but are not limited to BYL719, GSK2636771, TGX-221, AS25242, CAL-101, ZSTK474, (2-(difluoromethyl)-1-(4,6-di-4-morpholinyl-1,3,5-triazin-2-yl)-1H-benzimidazole; CAS #475110-96-4) and IPI-145.

Further illustrative examples of pan-PI3K inhibitors include, but are not limited to BEZ235, LY294002, GSK1059615, and GDC-0941.

In a preferred embodiment, a population of immune effector cells comprising T cells is transduced with a retroviral vector in the presence of poloxamer 407 and ZSTK474.

### D. Engineered Antigen Receptors

In particular embodiments, an immune effector cell is transduced with a retroviral vector or lentiviral vector encoding an engineered antigen receptor in the presence of a poloxamer. In particular embodiments, the engineered antigen receptor is an engineered T cell receptor (TCR), a chimeric antigen receptor (CAR), a DARIC receptor or components thereof, or a zetakine.

1. Engineered TCRs

In particular embodiments, an immune effector cell is transduced with a retroviral vector or lentiviral vector encoding an engineered TCR in the presence of a poloxamer and optionally a PI3K inhibitor. Naturally occurring T cell receptors comprise two subunits, an alpha chain and a beta chain subunit, each of which is a unique protein produced by recombination event in each T cell's genome. Libraries of TCRs may be screened for their selectivity to particular target antigens. In this manner, natural TCRs, which have a high-avidity and reactivity toward target antigens may be selected, cloned, and subsequently introduced into a population of T cells used for adoptive immunotherapy.

In one embodiment, T cells are modified by introducing a TCR subunit that has the ability to form TCRs that confer specificity to T cells for tumor cells expressing a target antigen. In particular embodiments, the subunits have one or more amino acid substitutions, deletions, insertions, or modifications compared to the naturally occurring subunit, so long as the subunits retain the ability to form TCRs and confer upon transfected T cells the ability to home to target cells, and participate in immunologically-relevant cytokine signaling. The engineered TCRs preferably also bind target cells displaying the relevant tumor-associated peptide with high avidity, and optionally mediate efficient killing of target cells presenting the relevant peptide in vivo.

The nucleic acids encoding engineered TCRs are preferably isolated from their natural context in a (naturally-occurring) chromosome of a T cell, and can be incorporated into suitable vectors as described elsewhere herein. Both the nucleic acids and the vectors comprising them can be transferred into a cell, preferably a T cell in particular embodiments. The modified T cells are then able to express one or more chains of a TCR encoded by the transduced nucleic acid or nucleic acids. In preferred embodiments, the engineered TCR is an exogenous TCR because it is introduced into T cells that do not normally express the particular TCR. The essential aspect of the engineered TCRs is that it has high avidity for a tumor antigen presented by a major histocompatibility complex (MHC) or similar immunological component. In contrast to engineered TCRs, CARs are engineered to bind target antigens in an MHC independent manner.

Antigens that are recognized by the engineered TCRs contemplated in particular embodiments include, but are not limited to cancer antigens, including antigens on both hematological cancers and solid tumors. Illustrative antigens include, but are not limited to alpha folate receptor, alpha folate receptor, 5T4, αvβ6 integrin, BCMA, B7-H3, B7-H6, CAIX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, VEGFR2, and WT-1.

In one embodiment, an immune effector cell is transduced with a retroviral vector or lentiviral vector encoding an engineered TCR in the presence of poloxamer 407 and ZSTK474.

2. Chimeric Antigen Receptors

In various embodiments, immune effector cells are transduced with retroviral or lentiviral vectors encoding a CAR that redirects cytotoxicity toward tumor cells in the presence of a poloxamer and optionally a PI3K inhibitor. CARS are molecules that combine antibody-based specificity for a target antigen (e.g., tumor antigen) with a T cell receptor-activating intracellular domain to generate a chimeric protein that exhibits a specific anti-tumor cellular immune activity. As used herein, the term, “chimeric,” describes being composed of parts of different proteins or DNAs from different origins.

In various embodiments, a CAR comprises an extracellular domain that binds to a specific target antigen (also referred to as a binding domain or antigen-specific binding domain), a transmembrane domain and an intracellular signaling domain. The main characteristic of CARs is their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific coreceptors.

In particular embodiments, CARs comprise an extracellular binding domain that specifically binds to a target polypeptide, e.g., target antigen, expressed on tumor cell. In particular embodiments, the extracellular binding domain comprises an antibody or antigen binding fragment thereof.

In one preferred embodiment, the binding domain is an scFv.

In particular embodiments, the CAR comprises an extracellular domain that binds an antigen selected from the group consisting of: alpha folate receptor, 5T4, αvβ6 integrin, BCMA, B7-H3, B7-H6, CAIX, CD16, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, VEGFR2, and WT-1.

In one embodiment, a CAR comprises a spacer domain comprising the CH2 and CH3 regions of IgG1, IgG4, or IgD.

In one embodiment, the binding domain of the CAR is linked to one or more hinge domains. Illustrative hinge domains suitable for use in the CARs described herein include the hinge region derived from the extracellular regions of type 1 membrane proteins such as CD8α, and CD4, which may be wild-type hinge regions from these molecules or may be altered. In another embodiment, the hinge domain comprises a CD8a hinge region.

In one embodiment, the hinge is a PD-1 hinge or CD152 hinge.

In one embodiment, a CAR comprises a TM domain derived from (i.e., comprise at least the transmembrane region(s) of the alpha or beta chain of the T-cell receptor, CD3δ, CD3ε, CD3γ, CD3ζ, CD4, CD5, CD8α, CD9, CD 16, CD22, CD27, CD28, CD33, CD37, CD45, CD64, CD80, CD86, CD 134, CD137, CD152, CD154, AMN, and PD-1.

In one embodiment, a CAR comprises a TM domain derived from CD8α. In another embodiment, a CAR contemplated herein comprises a TM domain derived from CD8α and a short oligo- or polypeptide linker, preferably between 1, 2, 3, 4, 5, 6, 7, 8, 9, or 10 amino acids in length that links the TM domain and the intracellular signaling domain of the CAR. A glycine-serine linker provides a particularly suitable linker.

In particular embodiments, a CAR comprises an intracellular signaling domain.

In preferred embodiments, a CAR comprises an intracellular signaling domain that comprises one or more costimulatory signaling domains and a primary signaling domain.

Primary signaling domains regulate primary activation of the TCR complex either in a stimulatory way, or in an inhibitory way. Primary signaling domains that act in a stimulatory manner may contain signaling motifs which are known as immunoreceptor tyrosine-based activation motifs or ITAMs.

Illustrative examples of ITAM containing primary signaling domains suitable for use in CARs contemplated in particular embodiments include those derived from FcRγ, FcRβ, CD3γ, CD3δ, CD3ε, CD3ζ, CD22, CD79a, CD79b, and CD66d. In particular preferred embodiments, a CAR comprises a CD3ζ primary signaling domain and one or more costimulatory signaling domains. The intracellular primary signaling and costimulatory signaling domains may be linked in any order in tandem to the carboxyl terminus of the transmembrane domain.

In particular embodiments, a CAR comprises one or more costimulatory signaling domains. Illustrative examples of such costimulatory molecules suitable for use in CARS contemplated in particular embodiments include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70. In one embodiment, a CAR comprises one or more costimulatory signaling domains selected from the group consisting of CD28, CD137, and CD134, and a CD3 primary signaling domain.

In various embodiments, the CAR comprises: an extracellular domain that binds an antigen selected from the group consisting of: BCMA, CD19, CSPG4, PSCA, ROR1, and TAG72; a transmembrane domain isolated from a polypeptide selected from the group consisting of: CD4, CD8α, CD154, and PD-1; one or more intracellular costimulatory signaling domains isolated from a polypeptide selected from the group consisting of: CD28, CD134, and CD137; and a signaling domain isolated from a polypeptide selected from the group consisting of: FcRγ, FcRβ, CD3γ, CD3δ, CD3ε, CD3ζ, CD22, CD79a, CD79b, and CD66d.

In one embodiment, an immune effector cell is transduced with a retroviral vector or lentiviral vector encoding a chimeric antigen receptor in the presence of poloxamer 407 and ZSTK474.

3. DARIC

In various embodiments, immune effector cells are transduced with retroviral or lentiviral vectors encoding one or more chains of a DARIC receptor in the presence of a poloxamer and optionally a PI3K inhibitor. As used herein, the term “DARIC receptor” refers to a multi-chain engineered antigen receptor. Illustrative examples of DARIC architectures and components are disclosed in PCT Publication No. WO2015/017214 and U.S. Patent Publication No. 20150266973, each of which is incorporated here by reference in its entirety.

In one embodiment, a retroviral vector encodes the following DARIC components: a signaling polypeptide comprising a first multimerization domain, a first transmembrane domain, and one or more intracellular co-stimulatory signaling domains and/or primary signaling domains; and a binding polypeptide comprising a binding domain, a second multimerization domain, and optionally a second transmembrane domain. A functional DARIC comprises a bridging factor that promotes the formation of a DARIC receptor complex on the cell surface with the bridging factor associated with and disposed between the multimerization domains of the signaling polypeptide and the binding polypeptide.

In particular embodiments, the first and second multimerization domains associate with a bridging factor selected from the group consisting of: rapamycin or a rapalog thereof, coumermycin or a derivative thereof, gibberellin or a derivative thereof, abscisic acid (ABA) or a derivative thereof, methotrexate or a derivative thereof, cyclosporin A or a derivative thereof, FKCsA or a derivative thereof, trimethoprim (Tmp)-synthetic ligand for FKBP (SLF) or a derivative thereof, and any combination thereof.

Illustrative examples of rapamycin analogs (rapalogs) include, but are not limited to those disclosed in U.S. Pat. No. 6,649,595, everolimus, novolimus, pimecrolimus, ridaforolimus, tacrolimus, temsirolimus, umirolimus, and zotarolimus.

In certain embodiments, the first and second multimerization domains are a pair selected from FKBP and FRB, FKBP and calcineurin, FKBP and cyclophilin, FKBP and bacterial DHFR, calcineurin and cyclophilin, PYL1 and ABI1, or GIB1 and GAI, or variants thereof.

In one embodiment, the first multimerization domain comprises FRB T2098L, the second multimerization domain comprises FKBP12, and the bridging factor is rapalog AP21967.

In another embodiment, the first multimerization domain comprises FRB, the second multimerization domain comprises FKBP12, and the bridging factor is Rapamycin, temsirolimus or everolimus.

In particular embodiments, a signaling polypeptide a first transmembrane domain and a binding polypeptide comprises a second transmembrane domain or GPI anchor. Illustrative examples of the first and second transmembrane domains are isolated from a polypeptide independently selected from the group consisting of: CD3δ, CD3ε, CD3γ, CD3ζ, CD4, CD5, CD8α, CD9, CD 16, CD22, CD27, CD28, CD33, CD37, CD45, CD64, CD80, CD86, CD 134, CD137, CD152, CD154, AMN, and PD-1.

In one embodiment, a signaling polypeptide comprises one or more intracellular co-stimulatory signaling domains and/or primary signaling domains.

Illustrative examples of primary signaling domains suitable for use in DARIC signaling components contemplated in particular embodiments include those derived from FcRγ, FcRβ, CD3γ, CD3δ, CD3ε, CD3 CD22, CD79a, CD79b, and CD66d. In particular preferred embodiments, a DARIC signaling component comprises a CD3ζ primary signaling domain and one or more costimulatory signaling domains. The intracellular primary signaling and costimulatory signaling domains may be linked in any order in tandem to the carboxyl terminus of the transmembrane domain.

Illustrative examples of such costimulatory molecules suitable for use in DARIC signaling components contemplated in particular embodiments include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70. In one embodiment, a DARIC signaling component comprises one or more costimulatory signaling domains selected from the group consisting of CD28, CD137, and CD134, and a CD3ζ primary signaling domain.

In particular embodiments, a DARIC binding component comprises a binding domain. In one embodiment, the binding domain is an antibody or antigen binding fragment thereof.

In particular embodiments, the DARIC binding component comprises an extracellular domain that binds an antigen selected from the group consisting of: alpha folate receptor, 5T4, αvβ6 integrin, BCMA, B7-H3, B7-H6, CAIX, CD16, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, VEGFR2, and WT-1.

In one embodiment, a DARIC comprises a signaling polypeptide comprises a first multimerization domain of FRB T2098L, a CD8 transmembrane domain, a 4-1BB costimulatory domain, and a CD3 primary signaling domain; the binding polypeptide comprises an scFv that binds CD19, a second multimerization domain of FKBP12 and a CD4 transmembrane domain; and the bridging factor is rapalog AP21967.

In one embodiment, a DARIC comprises a signaling polypeptide comprises a first multimerization domain of FRB, a CD8 transmembrane domain, a 4-1BB costimulatory domain, and a CD3ζ primary signaling domain; the binding polypeptide comprises an scFv that binds CD19, a second multimerization domain of FKBP12 and a CD4 transmembrane domain; and the bridging factor is Rapamycin, temsirolimus or everolimus.

In one embodiment, an immune effector cell is transduced with a retroviral vector or lentiviral vector encoding one or more components of a DARIC receptor in the presence of poloxamer 407 and ZSTK474.

4. Zetakines

In various embodiments, immune effector cells are transduced with retroviral or lentiviral vectors encoding a zetakine in the presence of a poloxamer and optionally a PI3K inhibitor. Zetakines are chimeric transmembrane immunoreceptors that comprise an extracellular domain comprising a soluble receptor ligand linked to a support region capable of tethering the extracellular domain to a cell surface, a transmembrane region and an intracellular signaling domain. Zetakines, when expressed on the surface of T lymphocytes, direct T cell activity to those cells expressing a receptor for which the soluble receptor ligand is specific. Zetakine chimeric immunoreceptors redirect the antigen specificity of T cells, with application to treatment of a variety of cancers, particularly via the autocrine/paracrine cytokine systems utilized by human malignancy.

In particular embodiments, the zetakine comprises an immunosuppressive cytokine or cytokine receptor binding variant thereof, a linker, a transmembrane domain, and an intracellular signaling domain.

In particular embodiments, the cytokine or cytokine receptor binding variant thereof is selected from the group consisting of: interleukin-4 (IL-4), interleukin-6 (IL-6), interleukin-8 (IL-8), interleukin-10 (IL-10), and interleukin-13 (IL-13).

In certain embodiments, the linker comprises a CH2CH3 domain, hinge domain, or the like. In one embodiment, a linker comprises the CH2 and CH3 domains of IgG1, IgG4, or IgD. In one embodiment, a linker comprises a CD8α or CD4 hinge domain.

In particular embodiments, the transmembrane domain is selected from the group consisting of: the alpha or beta chain of the T-cell receptor, CD3δ, CD3ε, CD3γ, CD3ζ, CD4, CD5, CD8α, CD9, CD 16, CD22, CD27, CD28, CD33, CD37, CD45, CD64, CD80, CD86, CD 134, CD137, CD152, CD154, AMN, and PD-1.

In particular embodiments, the intracellular signaling domain is selected from the group consisting of: an ITAM containing primary signaling domain and/or a costimulatory domain.

In particular embodiments, the intracellular signaling domain is selected from the group consisting of: FcRγ, FcRβ, CD3γ, CD3δ, CD3ε, CD3 CD22, CD79a, CD79b, and CD66d.

In particular embodiments, the intracellular signaling domain is selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70.

In one embodiment, a chimeric cytokine receptor comprises one or more costimulatory signaling domains selected from the group consisting of CD28, CD137, and CD134, and a CD3 primary signaling domain.

In one embodiment, an immune effector cell is transduced with a retroviral vector or lentiviral vector encoding a zetakine in the presence of poloxamer 407 and ZSTK474.

### E. Compositions and Formulations

The formulations and compositions contemplated herein may comprise a combination of any number of transduced or non-transduced immune effector cells or a combination thereof, viral vectors, polypeptides, polynucleotides, and one or more agents that increase transduction efficiency and/or VCN, e.g., poloxamers and PI3K inhibitors, as described herein, formulated in pharmaceutically-acceptable or physiologically-acceptable solutions (e.g., culture medium), either alone, or in combination with one or more other modalities of therapy. Compositions include, without limitation, cultures contemplated herein, and in particular embodiments, the terms composition and culture may be used synonymously.

Particular ex vivo and in vitro formulations and compositions contemplated herein may comprise a combination of transduced or non-transduced immune effector cells or a combination thereof, viral vectors, and one or more agents that increase transduction efficiency and/or VCN, e.g., poloxamers and PI3K inhibitors, as described herein, formulated in pharmaceutically-acceptable or physiologically-acceptable solutions (e.g., culture medium) for administration to a cell, tissue, organ, or an animal, either alone, or in combination with one or more other modalities of therapy.

Particular in vivo formulations and compositions contemplated herein may comprise a combination of viral vectors, and one or more agents that increase transduction efficiency and/or VCN, e.g., poloxamers and PI3K inhibitors, as described herein, formulated in pharmaceutically-acceptable or physiologically-acceptable solutions (e.g., culture medium) for administration to a cell, tissue, organ, or an animal, either alone, or in combination with one or more other modalities of therapy.

In certain embodiments, compositions contemplated herein comprise a population of immune cells comprising a therapeutically-effective amount of transduced cells, as described herein, formulated together with one or more pharmaceutically acceptable carriers (additives) and/or diluents (e.g., pharmaceutically acceptable cell culture medium).

In certain other embodiments, the present invention provides compositions comprising a retroviral vector and one or more agents that increase transduction efficiency and/or VCN, e.g., poloxamers and PI3K inhibitors, as described herein, formulated together with one or more pharmaceutically acceptable carriers (additives) and/or diluents (e.g., pharmaceutically acceptable cell culture medium).

In certain other embodiments, the present invention provides compositions comprising a retroviral vector and poloxamer 407, and optionally a PI3K inhibitor, formulated together with one or more pharmaceutically acceptable carriers (additives) and/or diluents (e.g., pharmaceutically acceptable cell culture medium).

In particular embodiments, compositions comprise a population of cells therapeutic immune effector cells, a retroviral vector and one or more agents that increase transduction efficiency and/or VCN, e.g., poloxamers and PI3K inhibitors, as described herein, formulated together with one or more pharmaceutically acceptable carriers (additives) and/or diluents (e.g., pharmaceutically acceptable cell culture medium). In a related embodiment, the population of therapeutic cells comprises T cells.

In one embodiment, the population of cells comprises immune effector cells.

An “immune effector cell,” is any cell of the immune system that has one or more effector functions (e.g., cytotoxic cell killing activity, secretion of cytokines, induction of ADCC and/or CDC). The illustrative immune effector cells contemplated herein are T lymphocytes, in particular cytotoxic T cells (CTLs; CD8+ T cells), TILs, and helper T cells (HTLs; CD4+ T cells. In one embodiment, immune effector cells include natural killer (NK) cells. In one embodiment, immune effector cells include natural killer T (NKT) cells. Immune effector cells can be autologous/autogeneic (“self”) or non-autologous (“non-self,” e.g., allogeneic, syngeneic or xenogeneic).

Illustrative immune effector cells include T lymphocytes. The terms “T cell” or “T lymphocyte” are art-recognized and are intended to include thymocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), CD4+CD8+ T cell, CD4⁻CD8⁻ T cell, or any other subset of T cells. Other illustrative populations of T cells suitable for use in particular embodiments include naïve T cells and memory T cells.

Pharmaceutical compositions contemplated herein comprise transduced cells produced according to methods described herein and a pharmaceutically acceptable carrier.

In other embodiments, pharmaceutical compositions comprise a retroviral vector and agents that increase transduction efficiency and/or VCN: poloxamers and a PI3K inhibitor, as described herein.

The phrase “pharmaceutically acceptable” refers to molecular entities and compositions that do not produce an allergic or similar untoward reaction when administered to a human. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the therapeutic cells are administered. Illustrative examples of pharmaceutical carriers can be sterile liquids, such as cell culture media, water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients in particular embodiments, include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Except insofar as any conventional media or agent is incompatible with the active ingredient, its use in the therapeutic compositions is contemplated. Supplementary active ingredients can also be incorporated into the compositions.

In one embodiment, a composition comprising a carrier is suitable for parenteral administration, e.g., intravascular (intravenous or intraarterial), intraperitoneal or intramuscular administration. Pharmaceutically acceptable carriers include sterile aqueous solutions, cell culture media, or dispersions. The use of such media and agents for pharmaceutically active substances is well known in the art. Except insofar as any conventional media or agent is incompatible with the transduced cells, use thereof in the pharmaceutical compositions is contemplated.

In particular embodiments, compositions contemplated herein comprise genetically modified immune effector cells and a pharmaceutically acceptable carrier, e.g., pharmaceutically acceptable cell culture medium. A composition comprising a cell-based composition contemplated herein can be administered separately by enteral or parenteral administration methods or in combination with other suitable compounds to effect the desired treatment goals.

The pharmaceutically acceptable carrier must be of sufficiently high purity and of sufficiently low toxicity to render it suitable for administration to the human subject being treated. It further should maintain or increase the stability of the composition. The pharmaceutically acceptable carrier can be liquid or solid and is selected, with the planned manner of administration in mind, to provide for the desired bulk, consistency, etc., when combined with other components of the composition. For example, the pharmaceutically acceptable carrier can be, without limitation, a binding agent (e.g., pregelatinized maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyaciylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulfate, etc.). Other suitable pharmaceutically acceptable carriers for the compositions contemplated herein include, but are not limited to, water, salt solutions, alcohols, polyethylene glycols, gelatins, amyloses, magnesium stearates, talcs, silicic acids, viscous paraffins, hydroxymethylcelluloses, polyvinylpyrrolidones and the like.

Such carrier solutions also can contain buffers, diluents and other suitable additives. The term “buffer” as used herein refers to a solution or liquid whose chemical makeup neutralizes acids or bases without a significant change in pH. Examples of buffers contemplated herein include, but are not limited to, Dulbecco's phosphate buffered saline (PBS), Ringer's solution, 5% dextrose in water (D5W), normal/physiologic saline (0.9% NaCl).

The pharmaceutically acceptable carriers and/or diluents may be present in amounts sufficient to maintain a pH of the therapeutic composition of about 7. Alternatively, the therapeutic composition has a pH in a range from about 6.8 to about 7.4, e.g., 6.8, 6.9, 7.0, 7.1, 7.2, 7.3, and 7.4. In still another embodiment, the therapeutic composition has a pH of about 7.4.

Compositions contemplated herein may comprise a nontoxic pharmaceutically acceptable medium. The compositions may be a suspension. The term “suspension” as used herein refers to non-adherent conditions in which cells are not attached to a solid support. For example, cells maintained as a suspension may be stirred or agitated and are not adhered to a support, such as a culture dish.

In particular embodiments, compositions contemplated herein are formulated in a suspension, where the immune effector cells are dispersed within an acceptable liquid medium or solution, e.g., saline or serum-free medium, in an intravenous (IV) bag or the like. Acceptable diluents include, but are not limited to water, PlasmaLyte, Ringer's solution, isotonic sodium chloride (saline) solution, serum-free cell culture medium, and medium suitable for cryogenic storage, e.g., Cryostor® medium.

In certain embodiments, a pharmaceutically acceptable carrier is substantially free of natural proteins of human or animal origin, and suitable for storing a composition comprising a population of cells. The therapeutic composition is intended to be administered into a human patient, and thus is substantially free of cell culture components such as bovine serum albumin, horse serum, and fetal bovine serum.

In some embodiments, compositions are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions includes, but is not limited to QBSF-60 (Quality Biological, Inc.), StemPro-34 (Life Technologies), and X-VIVO 10.

In a preferred embodiment, the compositions comprising immune effector cells are formulated in PlasmaLyte.

In various embodiments, compositions comprising immune effector cells are formulated in a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CS5, and CryoStor CS2.

In one embodiment, the compositions are formulated in a solution comprising 50:50 PlasmaLyte A to CryoStor CS10.

In particular embodiments, the composition is substantially free of mycoplasma, endotoxin, and microbial contamination. By “substantially free” with respect to endotoxin is meant that there is less endotoxin per dose of cells than is allowed by the FDA for a biologic, which is a total endotoxin of 5 EU/kg body weight per day, which for an average 70 kg person is 350 EU per total dose of cells. In particular embodiments, compositions comprising immune effector cells transduced with a retroviral vector contemplated herein contains about 0.5 EU/mL to about 5.0 EU/mL, or about 0.5 EU/mL, 1.0 EU/mL, 1.5 EU/mL, 2.0 EU/mL, 2.5 EU/mL, 3.0 EU/mL, 3.5 EU/mL, 4.0 EU/mL, 4.5 EU/mL, or 5.0 EU/mL.

In certain embodiments, compositions and formulations suitable for the delivery of viral vector systems (i.e., viral-mediated transduction) are contemplated including, but not limited to, retroviral (e.g., lentiviral) vectors.

Exemplary formulations for ex vivo delivery may also include the use of various transfection agents known in the art, such as calcium phosphate, electroporation, heat shock and various liposome formulations (i.e., lipid-mediated transfection). Liposomes, as described in greater detail below, are lipid bilayers entrapping a fraction of aqueous fluid. DNA spontaneously associates to the external surface of cationic liposomes (by virtue of its charge) and these liposomes will interact with the cell membrane.

In particular embodiments, compositions contemplated herein may comprise one or more polypeptides, polynucleotides, vectors comprising same, agents that increase transduction efficiency and/or VCN, as described herein, and transduced cells, etc., formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules or various pharmaceutically-active agents. There is virtually no limit to other components that may also be included in the compositions in particular embodiments, provided that the additional agents do not adversely affect the ability of the composition to deliver the intended gene therapy.

In particular embodiments, formulation of pharmaceutically-acceptable excipients and carrier solutions is well-known to those of skill in the art, as is the development of suitable dosing and treatment regimens for using the particular compositions described herein in a variety of treatment regimens, including e.g., enteral and parenteral, e.g., intravascular, intravenous, intrarterial, intraosseously, and intramedullary administration and formulation. It would be understood by the skilled artisan that particular embodiments contemplated herein may comprise other formulations, such as those that are well known in the pharmaceutical art, and are described, for example, in Remington: The Science and Practice of Pharmacy, 20th Edition. Baltimore, Md.: Lippincott Williams & Wilkins, 2005, which is incorporated by reference herein, in its entirety.

### F. Cell Culture Compositions

As discussed herein throughout, in particular embodiments, compositions and methods contemplated herein are useful for ex vivo and in vivo adoptive immunotherapies comprising a population of genetically modified T cells. In particular embodiments, compositions may comprise cells in culture, i.e., a cell culture composition. A cell culture composition may comprise a population of cells comprising immune effector, a suitable cell culture medium, and one or more poloxamers. A cell culture composition may comprise a population of cells comprising immune effector cells, a suitable cell culture medium, poloxamer 407 an optionally, one or more PI3K inhibitors.

In particular embodiments, cultured cells are immune effector cells or T cells.

In one embodiment, a cell culture composition comprises a population of cells comprising immune effector cells, a cell culture medium suitable for human administration, and a poloxamer. In one embodiment, a cell culture composition comprises a population of cells comprising immune effector cells, a cell culture medium suitable for human administration, a poloxamer and aPI3K inhibitor.

In one embodiment, a cell culture composition comprises a population of cells comprising genetically modified immune effector cells, a cell culture medium suitable for administration to a human, and a poloxamer.

In one embodiment, a cell culture composition comprises a population of cells comprising genetically modified immune effector cells, a cell culture medium suitable for administration to a human, a poloxamer and a PI3K inhibitor.

In some embodiments, the cell culture medium is a pharmaceutically acceptable cell culture medium.

Cell culture compositions contemplated herein, that comprise transduced immune effector cells, can be administered systemically or by directed injection to a subject in need thereof in order to effect the desired therapy.

### G. Transduction Methods

Methods and compositions contemplated herein significantly increase the transduction efficiency (TE) and vector copy number (VCN) of target cells. Without wishing to be bound to any particular theory, it is contemplated that the compositions and methods contemplated herein may be used to increase the VCN and transduce significantly more cells with significantly less virus, thereby minimizing the risk of genomic alteration and/or insertional activation of proto-oncogenes in the genome of the therapeutic cell, while simultaneously increasing the therapeutic efficacy of the drug product produced. Thus, the compositions and methods contemplated herein not only lead to production of a safer therapy, but to a more robust and therapeutically efficacious drug product. In addition, more efficient transduction allows less virus to be used per transduction and thus, decreases the cost of goods for adoptive immunotherapies comprising genetically modified cells.

The delivery of a gene(s) or other polynucleotide sequences using a retroviral or lentiviral vector by means of viral infection rather than by transfection is referred to as transduction. In one embodiment, retroviral vectors are transduced into a cell through infection and provirus integration. In certain embodiments, a cell, e.g., a target cell, is transduced if it comprises a gene or other polynucleotide sequence delivered to the cell by infection using a viral or retroviral vector. In particular embodiments, a transduced cell comprises one or more genes or other polynucleotide sequences delivered by a retroviral or lentiviral vector in its cellular genome.

In particular embodiments, host cells or target cells transduced with a viral vector express an engineered antigen receptor and are administered to a subject to treat and/or prevent a disease, disorder, or condition.

The production of infectious viral particles and viral stock solutions may be carried out using conventional techniques. Methods of preparing viral stock solutions are known in the art and are illustrated by, e.g., Y. Soneoka et al. (1995) Nucl. Acids Res. 23:628-633, and N. R. Landau et al. (1992) J. Virol. 66:5110-5113.

In particular embodiments, HIV type 1 (HIV-1) based viral particles may be generated by co-expressing the virion packaging elements and the transfer vector in a producer cell. These cells may be transiently transfected with a number of plasmids. Typically from three to five plasmids are employed, but the number may be greater depending upon the degree to which the lentiviral components are broken up into separate units. For example, one plasmid may encode the core and enzymatic components of the virion, derived from HIV-1. This plasmid is termed the packaging plasmid. Another plasmid typically encodes the envelope protein(s), most commonly the G protein of vesicular stomatitis virus (VSV G) because of its high stability and broad tropism. This plasmid may be termed the envelope expression plasmid. Yet another plasmid encodes the genome to be transferred to the target cell, that is, the vector itself, and is called the transfer vector. The packaging plasmids can be introduced into human cell lines by known techniques, including calcium phosphate transfection, lipofection or electroporation. Recombinant viruses with titers of several millions of transducing units per milliliter (TU/mL) can be generated by this technique and variants thereof. After ultracentrifugation concentrated stocks of about 10⁸ TU/mL, 10⁹ TU/mL, 10¹⁰ TU/mL, 10¹¹ TU/mL, 10¹² TU/mL, or about 10¹³ TU/mL can be obtained.

Infectious virus particles may be collected from the packaging cells using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art, e.g., Kutner et al., BMC Biotechnol. 2009; 9:10. doi: 10.1186/1472-6750-9-10; Kutner et al. Nat. Protoc. 2009; 4(4):495-505. doi: 10.1038/nprot.2009.22.

Viruses may be used to infect cells in vivo, ex vivo, or in vitro using techniques well known in the art. For example, when cells, for instance peripheral blood mononucleated cells (PBMCs) or T cells are transduced ex vivo, the vector particles may be incubated with the cells using a dose generally in the order of between 1 to 50 multiplicities of infection (MOI) which also corresponds to 1×10⁵ to 50×10⁵ transducing units of the viral vector per 10⁵ cells. This, of course, includes amount of vector corresponding to 1, 2, 3, 4, 5, 6, 7, 8, 9, 10, 15, 20, 25, 30, 35, 40, 45, and 50 MOI and all integer values in between.

In particular embodiments, retroviral or lentiviral vector is used at an MOI of about 10 to about 25 to transduce a population of cells.

In particular embodiments, retroviral or lentiviral vector is used at an MOI of about 10 to about 20 to transduce a population of cells.

In some embodiments, retroviral or lentiviral vector is used at an MOI of about 10, about 11, about 12, about 13, about 14, about 15, about 16, about 17, about 18, about 19, about 20, about 21, about 22, about 23, about 24, about 25, about 26, about 27, about 28, about 29 or about 30 to transduce a population of cells.

Viruses may also be delivered to a subject in vivo, by direct injection to the cell, tissue, or organ in need of therapy. Direct injection requires on the order of between 1 to 100 multiplicities of infection (MOI) which also corresponds to 1×10⁵ to 100×10⁵ transducing units of the viral vector per 10⁵ cells. This, of course, includes amount of vector corresponding to 1, 2, 3, 4, 5, 6, 7, 8, 9, 10, 15, 20, 25, 30, 35, 40, 45, 50, 55, 50, 65, 70, 75, 80, 85, 90, 95, and 100 MOI and all integer values in between.

Viruses may also be delivered according to viral titer (TU/mL), which can be measured, for example, by using a commercially available p24 titer assay, which is an ELISA against the p24 viral coat protein. The following formula can be used to calculate the pg/mL of p24: there are approximately 2000 molecules of p24 per physical particle (PP) of lentivirus: (2×10³)×(24×10³ Da of p24 per PP), 48×10⁶/Avogadro=(48×10⁶)/(6×10²³)=8×10⁻¹⁷ g of p24 per PP, approximately 1 PP per 1×10⁻¹⁶ g of p24, 1×10⁴ PP per pg of p24. A reasonably well packaged, VSV-G pseudotyped lentiviral vector will have an infectivity index in the range of 1 TU per 1000 physical particles (PP) to 1 TU per 100 PP (or less). Thus, the range is approximately 10 to 100 TU/pg of p24. It is through this conversion that TU/mL is obtained.

Based on previous experience, the amount of retroviral or lentivirus directly injected is determined by total TU and can vary based on both the volume that could be feasibly injected to the site and the type of tissue to be injected. For example, a bone marrow injection site may only allow for a very small volume of virus to be injected, so a high titer prep would be preferred, a TU of about 1×10⁶ to 1×10⁷, about 1×10⁶ to 1×10⁸, 1×10⁶ to 1×10⁹, about 1×10⁷ to 1×10¹⁰, 1×10⁸ to 1×10¹¹, about 1×10⁸ to 1×10¹², or about 1×10¹⁰ to 1×10¹² or more per injection could be used. However, a systemic delivery could accommodate a much larger TU, a load of 1×10⁸, 1×10⁹, 1×10¹⁰, 1×10¹¹, 1×10¹², 1×10¹³, 1×10¹⁴, or 1×10¹⁵, could be delivered.

Compositions and methods contemplated herein provide high transduction efficiency and VCN of immune effector cells in vitro, ex vivo, and in vivo, using lower viral titers than those disclosed above to achieve comparable transduction efficiencies in the absence of the compositions and methods provided herein.

Certain embodiments contemplated herein arise from the unexpected finding that transduction efficiency and/or VCN is significantly increased by contacting immune effector cells, in vitro, ex vivo, or in vivo, with a retrovirus and a poloxamer, e.g., poloxamer 407.

Illustrative final poloxamer concentrations used to transduced immune effector cells include, but are not limited to about 10 μg/mL to about 5000 μg/mL, about 10 μg/mL to about 2500 μg/mL, about 10 μg/mL to about 1000 μg/mL, about 50 μg/mL to about 1000 μg/mL, about 100 μg/mL to about 1000 μg/mL, about 200 μg/mL to about 1000 μg/mL, about 200 μg/mL to about 500 μg/mL, or about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, about 100 μg/mL, about 200 μg/mL, about 300 μg/mL, about 400 μg/mL, about 500 μg/mL, about 600 μg/mL, about 700 μg/mL, about 800 μg/mL, about 900 μg/mL, about 1000 μg/mL, about 1250 μg/mL, about 1500 μg/mL, about 1750 μg/mL, about 2000 μg/mL, about 2500 μg/mL, or about 5000 μg/mL or more, and any intervening concentration thereof.

In one embodiment, the agent is a PI3K inhibitor including but not limited to ZSTK474, as well as “analogs” or “derivatives” thereof.

Illustrative final PI3K concentrations used to transduce immune effector cells include, but are not limited to about 10 μM to about 200 μM, about 10 μM to about 100 μM, about 50 μM to about 100 μM, or about 10 μM, about 20 μM, about 30 μM, about 40 μM, about 50 μM, about 60 μM, about 70 μM, about 80 μM, about 90 μM, or about 100 μM or more, and any intervening concentration thereof.

In particular embodiments, immune effector cells may be cultured in the presence of a retrovirus may be exposed to (contacted with) a poloxamer and optionally a PI3K inhibitor, for a duration of about 10 minutes to about 72 hours, about 30 minutes to about 72 hours, about 30 minutes to about 48 hours, about 30 minutes to about 24 hours, about 30 minutes to about 12 hours, about 30 minutes to about 8 hours, about 30 minutes to about 6 hours, about 30 minutes to about 4 hours, about 30 minutes to about 2 hours, or about 1 hour to about 2 hours.

In particular embodiments, immune effector cells may be cultured in the presence of a retrovirus may be exposed to (contacted with) a poloxamer and optionally a PI3K inhibitor, for a duration of about 10 minutes, about 30 minutes, about 1 hour, about 2 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 48 hours, or about 72 hours, or any intervening duration of time.

In another embodiment, immune effector cells may be cultured with a retrovirus prior to culture with one or more agents that increases transduction efficiency and/or VCN, during culture with one or more agents that increases transduction efficiency and/or VCN, or after culture with one or more agents that increases transduction efficiency and/or VCN for any of the foregoing periods of time disclosed herein.

Furthermore, one having ordinary skill in the art would appreciate that in particular embodiments transduction efficiency and VCN are increased by culturing immune effector cells with retrovirus and a poloxamer and optionally a PI3K inhibitor, during the first 6 hours of transduction, the first 12 hours of transduction, the first 24 hours of transduction, the first 48 hours of transduction, or the first 72 hours of the transduction, or any intervening duration of transduction.

In particular embodiments, immune effector cells are transduced in a vessel, such as a bag, e.g., blood bag, and are agitated during the transduction.

As disclosed throughout, the compositions and methods contemplated herein offer unexpected increases in transduction efficiency and VCN of immune effector cells obtained from a patient having a cancer, infectious disease, autoimmune disease, inflammatory disease, and immunodeficiency, which are notoriously difficult to transduce and typically have low VCNs.

In various embodiments, the compositions and methods contemplated herein increase transduction efficiency to at least about 30%, at least about 40%, at least about 50%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, including any intervening percentages.

In various embodiments, the compositions and methods contemplated herein increase average VCN to at least about 0.5 to at least about 5.0, at least about 0.5 to at least about 3, at least about 0.5 to at least about 1.0, at least about 1.0 to at least about 5.0, at least about 1.0 to at least about 3.0, or at least about 0.5, at least about 1.0, at least about 1.5, at least about 2.0, at least about 2.5, at least about 3.0, at least about 3.5, at least about 4.0, at least about 4.5, or at least about 5.0.

In various embodiments, immune effector cells transduced with the compositions and methods contemplated herein have a transduction efficiency of at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% and an average VCN of at least about 0.5, at least about 1.0, at least about 1.5, at least about 2.0, or at least about 2.5.

In particular embodiments, an increase in transduction efficiency represents at least 2-fold, at least 5-fold, at least 10-fold, at least 25-fold, at least 50-fold, or at least 100-fold, or more fold enrichment of immune effector cells transduced with the compositions and methods contemplated herein compared to immune effector cells transduced with vector alone.

In particular embodiments, increase in average VCN represents at least 2-fold, at least 5-fold, at least 10-fold, at least 25-fold, at least 50-fold, or at least 100-fold, or more fold enrichment in VCN of immune effector cells transduced with the compositions and methods contemplated herein compared to immune effector cells transduced with vector alone.

Following transduction, the transduced cells may be cultured under conditions suitable for their maintenance, growth or proliferation. In particular embodiments, the transduced cells are cultured for about 1, 2, 3, 4, 5, 6, 7, 8, 9, 10, 11, 12, 13 or 14 days before transplantation.

Certain embodiments contemplate isolation and transduction of a population of cells. As used herein, the term “population of cells” refers to a plurality of cells that may be made up of any number and/or combination of homogenous or heterogeneous cell types, as described elsewhere herein. For example, for transduction of immune effector cells, a population of cells may be isolated or obtained from peripheral blood mononuclear cells, bone marrow, lymph nodes tissue, cord blood, thymus issue, tissue from a site of infection, ascites, pleural effusion, spleen tissue, and tumors. A population of cells may comprise about 10%, about 20%, about 30%, about 40%, about 50%, about 60%, about 70%, about 80%, about 90%, or about 100% of the target cell type to be transduced. In certain embodiments, immune effector cells may be isolated or purified from a population of heterogeneous cells using methods known in the art.

Preferred target cell types transduced with the compositions and methods contemplated herein include, immune effector cells, e.g., human immune effector cells.

In preferred embodiments, the compositions and methods contemplated herein are used to increase the transduction efficiency and/or VCN of immune effector r cells.

Illustrative sources to obtain immune effector cells transduced with the methods and compositions contemplated herein include, but are not limited to: peripheral blood mononuclear cells, bone marrow, lymph nodes tissue, cord blood, thymus issue, tissue from a site of infection, ascites, pleural effusion, spleen tissue, and tumors.

### H. Therapeutic Methods

Drug products comprising a higher proportion of transduced cells, wherein the copy number of the therapeutic genes in each cell is also higher provides for more therapeutically efficacious therapies. As used herein, the term “drug product” refers to genetically modified cells produced using the compositions and methods contemplated herein. In particular embodiments, the drug product comprises genetically modified immune effector cells, e.g., T cells. The modified T cells contemplated in particular embodiments provide safer and more efficacious adoptive cell therapies because they are resistant to T cell exhaustion and display increased durability and persistence in the tumor microenvironment that can lead to sustained therapy.

The immune effector cells, including T cells modified to express an engineered antigen receptor contemplated herein provide improved methods of adoptive immunotherapy for use in the prevention, treatment, and amelioration cancers, or for preventing, treating, or ameliorating at least one symptom associated with a cancer.

The immune effector cells that comprise an engineered receptor contemplated herein provide improved drug products for use in the prevention, treatment, or amelioration of at least one symptom of a cancer, GVHD, an infectious disease, an autoimmune disease, an inflammatory disease, or an immunodeficiency.

In particular embodiments, an effective amount of modified immune effector cells or T cells comprising an engineered receptor are administered to a subject to prevent, treat, or ameliorate at least one symptom of a cancer, GVHD, an infectious disease, an autoimmune disease, an inflammatory disease, or an immunodeficiency.

In particular embodiments, a method of preventing, treating, or ameliorating at least one symptom of a cancer comprises administering the subject an effective amount of modified immune effector cells or T cells comprising an engineered TCR, CAR, or DARIC, or other therapeutic transgene to redirect the cells to a tumor or cancer.

In particular embodiments, the modified immune effector cells contemplated herein are used in the treatment of solid tumors or cancers.

In particular embodiments, the modified immune effector cells contemplated herein are used in the treatment of solid tumors or cancers including, but not limited to: adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain/CNS cancer, breast cancer, bronchial tumors, cardiac tumors, cervical cancer, cholangiocarcinoma, chondrosarcoma, chordoma, colon cancer, colorectal cancer, craniopharyngioma, ductal carcinoma in situ (DCIS) endometrial cancer, ependymoma, esophageal cancer, esthesioneuroblastoma, Ewing's sarcoma, extracranial germ cell tumor, extragonadal germ cell tumor, eye cancer, fallopian tube cancer, fibrous histiosarcoma, fibrosarcoma, gallbladder cancer, gastric cancer, gastrointestinal carcinoid tumors, gastrointestinal stromal tumor (GIST), germ cell tumors, glioma, glioblastoma, head and neck cancer, hemangioblastoma, hepatocellular cancer, hypopharyngeal cancer, intraocular melanoma, kaposi sarcoma, kidney cancer, laryngeal cancer, leiomyosarcoma, lip cancer, liposarcoma, liver cancer, lung cancer, non-small cell lung cancer, lung carcinoid tumor, malignant mesothelioma, medullary carcinoma, medulloblastoma, menangioma, melanoma, Merkel cell carcinoma, midline tract carcinoma, mouth cancer, myxosarcoma, myelodysplastic syndrome, myeloproliferative neoplasms, nasal cavity and paranasal sinus cancer, nasopharyngeal cancer, neuroblastoma, oligodendroglioma, oral cancer, oral cavity cancer, oropharyngeal cancer, osteosarcoma, ovarian cancer, pancreatic cancer, pancreatic islet cell tumors, papillary carcinoma, paraganglioma, parathyroid cancer, penile cancer, pharyngeal cancer, pheochromocytoma, pinealoma, pituitary tumor, pleuropulmonary blastoma, primary peritoneal cancer, prostate cancer, rectal cancer, retinoblastoma, renal cell carcinoma, renal pelvis and ureter cancer, rhabdomyosarcoma, salivary gland cancer, sebaceous gland carcinoma, skin cancer, soft tissue sarcoma, squamous cell carcinoma, small cell lung cancer, small intestine cancer, stomach cancer, sweat gland carcinoma, synovioma, testicular cancer, throat cancer, thymus cancer, thyroid cancer, urethral cancer, uterine cancer, uterine sarcoma, vaginal cancer, vascular cancer, vulvar cancer, and Wilms Tumor.

In particular embodiments, the modified immune effector cells contemplated herein are used in the treatment of solid tumors or cancers including, without limitation, liver cancer, pancreatic cancer, lung cancer, breast cancer, bladder cancer, brain cancer, bone cancer, thyroid cancer, kidney cancer, or skin cancer.

In particular embodiments, the modified immune effector cells contemplated herein are used in the treatment of various cancers including but not limited to pancreatic, bladder, and lung.

In particular embodiments, the modified immune effector cells contemplated herein are used in the treatment of liquid cancers or hematological cancers.

In particular embodiments, the modified immune effector cells contemplated herein are used in the treatment of B-cell malignancies, including but not limited to: leukemias, lymphomas, and multiple myeloma.

In particular embodiments, the modified immune effector cells contemplated herein are used in the treatment of liquid cancers including, but not limited to leukemias, lymphomas, and multiple myelomas: acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CML), chronic myelomonocytic leukemia (CMML) and polycythemia vera, Hodgkin lymphoma, nodular lymphocyte-predominant Hodgkin lymphoma, Burkitt lymphoma, small lymphocytic lymphoma (SLL), diffuse large B-cell lymphoma, follicular lymphoma, immunoblastic large cell lymphoma, precursor B-lymphoblastic lymphoma, mantle cell lymphoma, marginal zone lymphoma, mycosis fungoides, anaplastic large cell lymphoma, Sézary syndrome, precursor T-lymphoblastic lymphoma, multiple myeloma, overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

Preferred cells for use in the methods contemplated herein include autologous/autogeneic (“self”) cells, preferably immune effector cells, more preferably T cells, and more preferably immune effector cells.

In particular embodiments, methods comprising administering a therapeutically effective amount of modified immune effector cells contemplated herein or a composition comprising the same, to a patient in need thereof, alone or in combination with one or more therapeutic agents, are provided. In certain embodiments, the cells are used in the treatment of patients at risk for developing a cancer, GVHD, an infectious disease, an autoimmune disease, an inflammatory disease, or an immunodeficiency. Thus, particular embodiments comprise the treatment or prevention or amelioration of at least one symptom of a cancer, an infectious disease, an autoimmune disease, an inflammatory disease, or an immunodeficiency comprising administering to a subject in need thereof, a therapeutically effective amount of the genome edited cells contemplated herein.

In one embodiment, a method of treating a cancer, GVHD, an infectious disease, an autoimmune disease, an inflammatory disease, or an immunodeficiency in a subject in need thereof comprises administering an effective amount, e.g., therapeutically effective amount of a composition comprising modified immune effector cells contemplated herein. The quantity and frequency of administration will be determined by such factors as the condition of the patient, and the type and severity of the patient's disease, although appropriate dosages may be determined by clinical trials.

In one illustrative embodiment, the effective amount of modified immune effector cells provided to a subject is at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, or at least 10×10⁶ cells/kg, or more cells/kg, including all intervening doses of cells.

In another illustrative embodiment, the effective amount of modified immune effector cells provided to a subject is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, or about 10×10⁶ cells/kg, or more cells/kg, including all intervening doses of cells.

In another illustrative embodiment, the effective amount of modified immune effector cells provided to a subject is from about 2×10⁶ cells/kg to about 10×10⁶ cells/kg, about 3×10⁶ cells/kg to about 10×10⁶ cells/kg, about 4×10⁶ cells/kg to about 10×10⁶ cells/kg, about 5×10⁶ cells/kg to about 10×10⁶ cells/kg, 2×10⁶ cells/kg to about 6×10⁶ cells/kg, 2×10⁶ cells/kg to about 7×10⁶ cells/kg, 2×10⁶ cells/kg to about 8×10⁶ cells/kg, 3×10⁶ cells/kg to about 6×10⁶ cells/kg, 3×10⁶ cells/kg to about 7×10⁶ cells/kg, 3×10⁶ cells/kg to about 8×10⁶ cells/kg, 4×10⁶ cells/kg to about 6×10⁶ cells/kg, 4×10⁶ cells/kg to about 7×10⁶ cells/kg, 4×10⁶ cells/kg to about 8×10⁶ cells/kg, 5×10⁶ cells/kg to about 6×10⁶ cells/kg, 5×10⁶ cells/kg to about 7×10⁶ cells/kg, 5×10⁶ cells/kg to about 8×10⁶ cells/kg, or 6×10⁶ cells/kg to about 8×10⁶ cells/kg, including all intervening doses of cells.

One of ordinary skill in the art would recognize that multiple administrations of the compositions contemplated in particular embodiments may be required to effect the desired therapy. For example, a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In certain embodiments, it may be desirable to administer activated T cells to a subject and then subsequently redraw blood (or have an apheresis performed), activate T cells therefrom, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, 100 cc, 150 cc, 200 cc, 250 cc, 300 cc, 350 cc, or 400 cc or more. Not to be bound by theory, using this multiple blood draw/multiple reinfusion protocol may serve to select out certain populations of T cells.

In one embodiment, a method of treating a subject diagnosed with a cancer, comprises removing immune effector cells from the subject, modifying the immune effector cells by introducing one or more vectors encoding an engineered antigen receptor and producing a population of modified immune effector cells, and administering the population of modified immune effector cells to the same subject. In a preferred embodiment, the immune effector cells comprise T cells.

The methods for administering the cell compositions contemplated in particular embodiments include any method which is effective to result in reintroduction of ex vivo modified immune effector cells or on reintroduction of the modified progenitors of immune effector cells that on introduction into a subject differentiate into mature immune effector cells. One method comprises modifying peripheral blood T cells ex vivo by introducing one or more vectors encoding an engineered antigen receptor and returning the transduced cells into the subject.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### F127 Increases Transduction Efficiency of T Cells with Lentivirus Encoding a CAR

Peripheral blood mononuclear cells (PBMC) were cultured in static flasks in media containing IL-2 (CellGenix), antibodies specific for CD3 and CD28 (Miltenyi Biotec). Activated PBMCs were transduced 24 hours later with a lentiviral vector encoding a chimeric antigen receptor (CAR) in 24-well plates (microculture) and in the presence of F127, F108 or both F127 and F108 at a concentration of about 1 mg/mL. After 12 days in culture, the VCN was determined using qPCR.

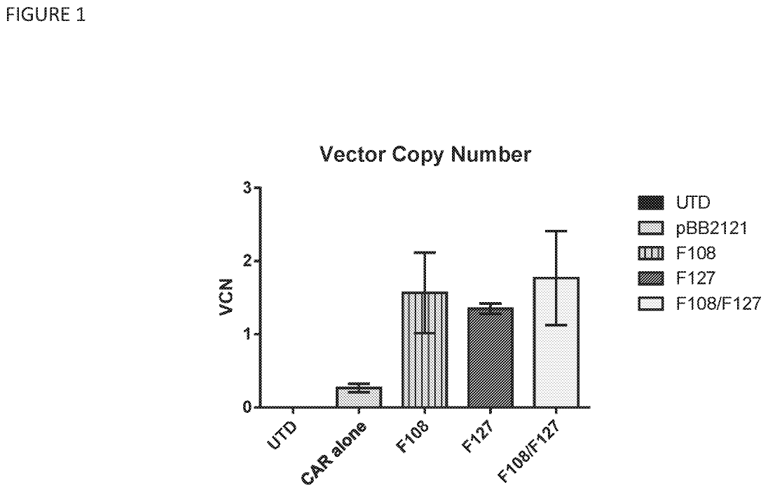

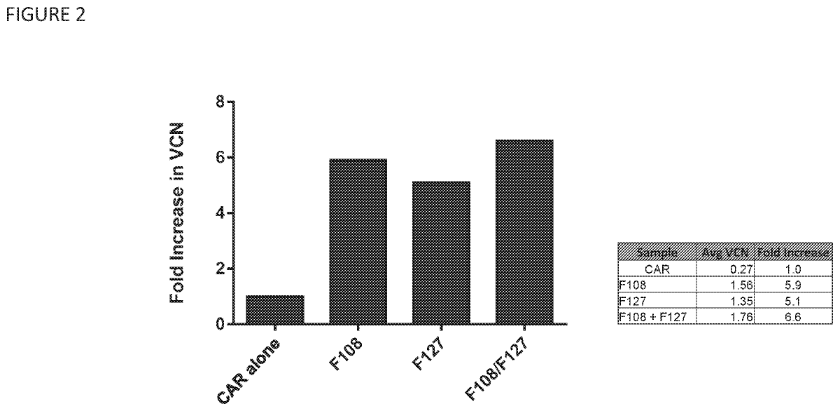

F127, F108, and F127 and F108 increased VCN from about 0.25 copies/diploid genome (c/dg) to about 1.35 to 1.75 c/dg. FIG. 1. In addition, F127, F108, and F127 and F108 increased VCN about 5- to 6-fold compared to the CAR only transduction control. FIG. 2.

### Example 2

### F127 Increases Transduction Efficiency of T Cells with Lentivirus Encoding an Engineered TCR

Peripheral blood mononuclear cells (PBMC) from three healthy donors were cultured in static flasks in media containing IL-2 (CellGenix), antibodies specific for CD3 and CD28 (Miltenyi Biotec). Activated PBMCs were transduced 24 hours later with a lentiviral vector encoding a transgenic NY-ESO-1 TCR in the presence of F127 or F108 at a concentration of about 1 mg/mL. After 10 days in culture, the vector copy number (VCN) was determined using qPCR and TCR expression was measure by staining for tetramers specific for the transgenic NY-ESO-1 TCR.

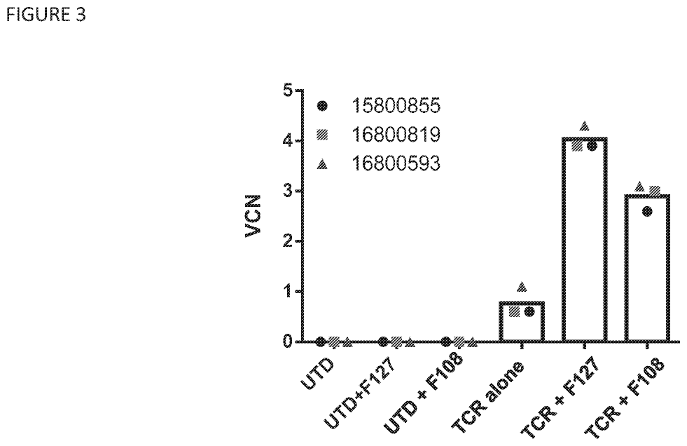

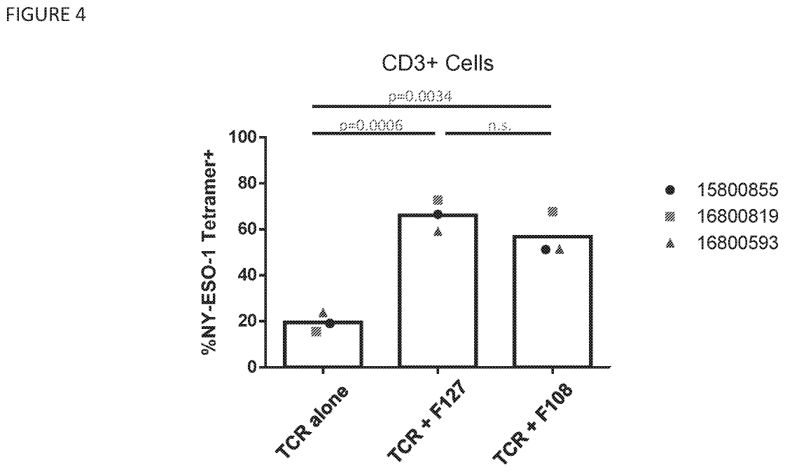

F127 increased VCN about 4-fold compared to transduction with the lentiviral vector control. FIG. 3. F108 also increased VCN, although to a lesser extent. Id. F127 also increased NY-ESO-1 transgenic TCR expression from 20% to 65% when compared to the lentiviral vector control. FIG. 4. F108 also increased NY-ESO-1 transgenic TCR expression, although to a lesser extent. Id.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A composition comprising a population of immune effector cells, a retroviral vector, and poloxamer 407.

2. The composition of claim 1, wherein the immune effector cells are isolated from a subject that has a cancer, an infectious disease, an autoimmune disease, an inflammatory disease, or an immunodeficiency.

3. The composition of claim 1 or claim 2, wherein the immune effector cells comprise CD3⁺ T cells, CD4⁺ T cells, and/or CD8⁺ T cells.

4. The composition of any one of claims 1-3, wherein the immune effector cells comprise cytotoxic T lymphocytes (CTLs), tumor infiltrating lymphocytes (TILs), or helper T cells.

5. The composition of claim 1 or claim 2, wherein the immune effector cells comprise natural killer (NK) cells or natural killer T (NKT) cells.

6. The composition of any one of claims 1-5, wherein the source of the immune effector cells is peripheral blood mononuclear cells, bone marrow, lymph nodes tissue, cord blood, thymus issue, tissue from a site of infection, ascites, pleural effusion, spleen tissue, or tumors.

7. The composition of any one of claims 1-6, wherein the retroviral vector encodes an engineered antigen receptor.

8. The composition of any one of claims 1-7, wherein the retroviral vector encodes an engineered antigen receptor selected from the group consisting of: an engineered T cell receptor (TCR), a chimeric antigen receptor (CAR), a DARIC receptor or components thereof, and a chimeric cytokine receptor.

9. The composition of claim 7 or claim 8, wherein the engineered antigen receptor comprises an extracellular binding domain that binds an antigen selected from the group consisting of: alpha folate receptor, 5T4, αᵥβ₆ integrin, BCMA, B7-H3, B7-H6, CAIX, CD16, CD19, CD20, CD22, CD30, CD33, CD37, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, VEGFR2, and WT-1.

10. The composition of claim 7 or claim 8, wherein the engineered antigen receptor comprises an extracellular binding domain that binds an antigen selected from the group consisting of: BCMA, CD19, CSPG4, PSCA, ROR1, and TAG72.

11. The composition of claim 7 or claim 8, wherein the engineered antigen receptor comprises an extracellular binding domain that binds an antigen selected from the group consisting of: BCMA, CD19, CD20, CD22, CD23, CD33, CD37, CD52, CD80, and HLA-DR.

12. The composition of claim 7 or claim 8, wherein the engineered antigen receptor comprises an extracellular binding domain that binds an antigen expressed on a cancer cell.

13. The composition of claim 7 or claim 8, wherein the engineered antigen receptor comprises an extracellular binding domain that binds an antigen expressed on a solid cancer cell.

14. The composition of claim 7 or claim 8, wherein the engineered antigen receptor comprises an extracellular binding domain that binds an antigen expressed on a liquid cancer cell.

15. The composition of claim 7 or claim 8, wherein the engineered antigen receptor comprises an extracellular binding domain that binds an antigen expressed on a malignant B cell.

16. The composition of claim 7 or claim 8, wherein the engineered antigen receptor comprises an extracellular binding domain that binds an antigen expressed on a malignant plasma cell.

17. The composition of any one of claims 1-16, wherein the retroviral vector is present at an MOI of about 10 to about 30.

18. The composition of any one of claims 1-16, wherein the retroviral vector is present at an MOI of about 10 to about 25.

19. The composition of any one of claims 1-16, wherein the retroviral vector is present at an MOI of about 10 to about 20.

20. The composition of any one of claims 1-16, wherein the retroviral vector is present at an MOI of about 10, about 11, about 12, about 13, about 14, about 15, about 16, about 17, about 18, about 19, about 20, about 21, about 22, about 23, about 24, about 25, about 26, about 27, about 28, about 29 or about 30.

21. The composition of any one of claims 1-20, wherein the retroviral vector is a lentiviral vector.

22. The composition of any one of claims 1-21, wherein the retroviral vector is derived from a lentivirus selected from the group consisting of: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

23. The composition of any one of claims 1-22, wherein the retroviral vector is derived from an HIV lentivirus.

24. The composition of any one of claims 1-23, wherein the retroviral vector is derived from an HIV-1 lentivirus.

25. The composition of any one of claims 1-24, wherein the population of cells comprises at least about 1×10⁶ T cells to about 1×10⁹ T cells.

26. The composition of any one of claims 1-24, wherein the population of cells comprises at least about 1×10⁷ T cells to about 1×10⁹ T cells.

27. The composition of any one of claims 1-24, wherein the population of cells comprises at least about 1×10⁸ T cells to about 1×10⁹ T cells.

28. The composition of any one of claims 1-24, wherein the population of cells comprises at least about 1×10⁶ T cells to about 1×10⁸ T cells.

29. The composition of any one of claims 1-24, wherein the population of cells comprises at least about 1×10⁶ T cells to about 1×10⁷ T cells.

30. The composition of any one of claims 1-24, wherein the population of cells comprises at least about 1×10⁶ T cells, at least about 1×10⁷T cells, at least about 1×10⁸ T cells, or at least about 1×10⁹ T cells.

31. The composition of any one of claims 1-30, wherein poloxamer 407 is present at a concentration of at least about 100 μg/mL.

32. The composition of any one of claims 1-30, wherein poloxamer 407 is present at a concentration of at least about 200 μg/mL.

33. The composition of any one of claims 1-30, wherein poloxamer 407 is present at a concentration of at least about 300 μg/mL.

34. The composition of any one of claims 1-30, wherein poloxamer 407 is present at a concentration of at least about 400 μg/mL.

35. The composition of any one of claims 1-30, wherein poloxamer 407 is present at a concentration of at least about 500 μg/mL.

36. The composition of any one of claims 1-35, further comprising a culture medium.

37. The composition of any one of claims 1-36, further comprising a PI3K inhibitor.

38. The composition of any one of claims 1-37, further comprising a PI3K inhibitor selected from the group consisting of: BEZ235, LY294002, TG100713, and GDC-0941.

39. The composition of any one of claims 1-37, further comprising a PI3K inhibitor selected from the group consisting of: BYL719, GSK2636771, TGX-221, AS25242, CAL-101, and IPI-145.

40. The composition of any one of claims 1-37, further comprising the PI3K inhibitor ZSTK474.

41. A population of therapeutic immune effector cells comprising immune effector cells isolated from a subject that has a cancer, an infectious disease, an autoimmune disease, an inflammatory disease, or an immunodeficiency, wherein at least 50% of the immune effector cells are transduced and viable and wherein the immune effector cells have an average vector copy number (VCN) of about 0.5 to 5.

42. The population of therapeutic immune effector cells of claim 41, wherein the retroviral vector transduced the immune effector cells at a multiplicity of infection (MOI) of about 10 to about 30.

43. The population of therapeutic immune effector cells of claim 41, wherein the retroviral vector transduced the immune effector cells at a multiplicity of infection (MOI) of about 10 to about 25.

44. The population of therapeutic immune effector cells of claim 41, wherein the retroviral vector transduced the immune effector cells at a multiplicity of infection (MOI) of about 10 to about 20.

45. The population of therapeutic immune effector cells of claim 41, wherein the retroviral vector transduced the immune effector cells at an MOI of about 10, about 11, about 12, about 13, about 14, about 15, about 16, about 17, about 18, about 19, about 20, about 21, about 22, about 23, about 24, about 25, about 26, about 27, about 28, about 29 or about 30.

46. The population of therapeutic immune effector cells of any one of claims 41-45, wherein at least 75% of the cells have been transduced.

47. The population of therapeutic immune effector cells of any one of claims 41-45, wherein at least 90% of the cells have been transduced.

48. The population of therapeutic immune effector cells of any one of claims 41-47, wherein the average VCN is at least 1.0.

49. The population of therapeutic immune effector cells of any one of claims 41-48, wherein the average VCN is at least 1.5.

50. The population of therapeutic immune effector cells of any one of claims 41-49, wherein the average VCN is at least 2.0.

51. The population of therapeutic immune effector cells of any one of claims 41-50, wherein the average VCN is at least 2.5.

52. The population of therapeutic immune effector cells of any one of claims 41-51, wherein the average VCN is at least 3.0.

53. The population of therapeutic immune effector cells of any one of claims 41-52, wherein viability of the population of cells is at least 75%.

54. The population of therapeutic immune effector cells of any one of claims 41-53, wherein viability of the population of cells is at least 85%.

55. The population of therapeutic immune effector cells of any one of claims 41-54, wherein viability of the population of cells is at least 95%.

56. The population of therapeutic immune effector cells of any one of claims 41-55, wherein the immune effector cells comprise CD3+ T cells, CD4+ T cells, and/or CD8⁺ T cells.

57. The population of therapeutic immune effector cells of any one of claims 41-56, wherein the immune effector cells comprise cytotoxic T lymphocytes (CTLs), tumor infiltrating lymphocytes (TILs), or helper T cells.

58. The population of therapeutic immune effector cells of any one of claims 41-55, wherein the immune effector cells comprise natural killer (NK) cells or natural killer T (NKT) cells.

59. The population of therapeutic immune effector cells of any one of claims 41-58, wherein the source of the immune effector cells is peripheral blood mononuclear cells, bone marrow, lymph nodes tissue, cord blood, thymus issue, tissue from a site of infection, ascites, pleural effusion, spleen tissue, or tumors.

60. The population of therapeutic immune effector cells of any one of claims 41-59, wherein the retroviral vector encodes an engineered antigen receptor.

61. The population of therapeutic immune effector cells of any one of claims 41-60, wherein the retroviral vector encodes an engineered antigen receptor selected from the group consisting of: an engineered T cell receptor (TCR), a chimeric antigen receptor (CAR), a DARIC receptor or components thereof, and a chimeric cytokine receptor.

62. The population of therapeutic immune effector cells of claim 60 or claim 61, wherein the engineered antigen receptor comprises an extracellular binding domain that binds an antigen selected from the group consisting of: alpha folate receptor, 5T4, αᵥβ₆ integrin, BCMA, B7-H3, B7-H6, CAIX, CD16, CD19, CD20, CD22, CD30, CD33, CD37, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, VEGFR2, and WT-1.

63. The population of therapeutic immune effector cells of claim 60 or claim 61, wherein the engineered antigen receptor comprises an extracellular binding domain that binds an antigen selected from the group consisting of: BCMA, CD19, CSPG4, PSCA, ROR1, and TAG72.

64. The population of therapeutic immune effector cells of claim 60 or claim 61, wherein the engineered antigen receptor comprises an extracellular binding domain that binds an antigen selected from the group consisting of: BCMA, CD19, CD20, CD22, CD23, CD33, CD37, CD52, CD80, and HLA-DR.

65. The population of therapeutic immune effector cells of claim 60 or claim 61, wherein the engineered antigen receptor comprises an extracellular binding domain that binds an antigen expressed on a cancer cell.

66. The population of therapeutic immune effector cells of claim 60 or claim 61, wherein the engineered antigen receptor comprises an extracellular binding domain that binds an antigen expressed on a solid cancer cell.

67. The population of therapeutic immune effector cells of claim 60 or claim 61, wherein the engineered antigen receptor comprises an extracellular binding domain that binds an antigen expressed on a liquid cancer cell.

68. The population of therapeutic immune effector cells of claim 60 or claim 61, wherein the engineered antigen receptor comprises an extracellular binding domain that binds an antigen expressed on a malignant B cell.

69. The population of therapeutic immune effector cells of claim 60 or claim 61, wherein the engineered antigen receptor comprises an extracellular binding domain that binds an antigen expressed on a malignant plasma cell.

70. The population of therapeutic immune effector cells of any one of claims 41-69, wherein the retroviral vector is a lentiviral vector.

71. The population of therapeutic immune effector cells of any one of claims 41-70, wherein the retroviral vector is derived from a lentivirus selected from the group consisting of: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

72. The population of therapeutic immune effector cells of any one of claims 41-71, wherein the retroviral vector is derived from an HIV lentivirus.

73. The population of therapeutic immune effector cells of any one of claims 41-72, wherein the retroviral vector is derived from an HIV-1 lentivirus.

74. The population of therapeutic immune effector cells of any one of claims 41-73, wherein the population of cells comprises at least about 1×10⁶ transduced T cells to about 1×10⁹ transduced T cells.

75. The population of therapeutic immune effector cells of any one of claims 41-73, wherein the population of cells comprises at least about 1×10⁷ transduced T cells to about 1×10⁹ transduced T cells.

76. The population of therapeutic immune effector cells of any one of claims 41-73, wherein the population of cells comprises at least about 1×10⁸ transduced T cells to about 1×10⁹ transduced T cells.

77. The population of therapeutic immune effector cells of any one of claims 41-73, wherein the population of cells comprises at least about 1×10⁶ transduced T cells to about 1×10⁸ transduced T cells.

78. The population of therapeutic immune effector cells of any one of claims 41-73, wherein the population of cells comprises at least about 1×10⁶ transduced T cells to about 1×10⁷ transduced T cells.

79. The population of therapeutic immune effector cells of any one of claims 41-73, wherein the population of cells comprises at least about 1×10⁶ transduced T cells, at least about 1×10⁷ transduced T cells, at least about 1×10⁸ transduced T cells, or at least about 1×10⁹ transduced T cells.

80. A method of transducing a population of immune effector cells comprising culturing the cells in a culture medium, in the presence of a retrovirus, and poloxamer 407.

81. The method of claim 80, wherein the immune effector cells are isolated from a subject that has a cancer, an infectious disease, an autoimmune disease, an inflammatory disease, or an immunodeficiency.

82. The method of claim 80 or claim 81, wherein the immune effector cells comprise CD3⁺ T cells, CD4⁺ T cells, and/or CD8⁺ T cells.

83. The method of any one of claims 80-82, wherein the immune effector cells comprise cytotoxic T lymphocytes (CTLs), tumor infiltrating lymphocytes (TILs), or helper T cells.

84. The method of claim 80 or claim 81, wherein the immune effector cells comprise natural killer (NK) cells or natural killer T (NKT) cells.

85. The method of any one of claims 80-84, wherein the source of the immune effector cells is peripheral blood mononuclear cells, bone marrow, lymph nodes tissue, cord blood, thymus issue, tissue from a site of infection, ascites, pleural effusion, spleen tissue, or tumors.

86. The method of any one of claims 80-85, wherein the retroviral vector encodes an engineered antigen receptor.

87. The method of any one of claims 80-86, wherein the retroviral vector encodes an engineered antigen receptor selected from the group consisting of: an engineered T cell receptor (TCR), a chimeric antigen receptor (CAR), a DARIC receptor or components thereof, and a chimeric cytokine receptor.

88. The method of claim 85 or claim 86, wherein the engineered antigen receptor comprises an extracellular binding domain that binds an antigen selected from the group consisting of: alpha folate receptor, 5T4, αᵥβ₆ integrin, BCMA, B7-H3, B7-H6, CAIX, CD16, CD19, CD20, CD22, CD30, CD33, CD37, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, VEGFR2, and WT-1.

89. The method of claim 85 or claim 86, wherein the engineered antigen receptor comprises an extracellular binding domain that binds an antigen selected from the group consisting of: BCMA, CD19, CSPG4, PSCA, ROR1, and TAG72.

90. The method of claim 85 or claim 86, wherein the engineered antigen receptor comprises an extracellular binding domain that binds an antigen selected from the group consisting of: BCMA, CD19, CD20, CD22, CD23, CD33, CD37, CD52, CD80, and HLA-DR.

91. The method of claim 85 or claim 86, wherein the engineered antigen receptor comprises an extracellular binding domain that binds an antigen expressed on a cancer cell.

92. The method of claim 85 or claim 86, wherein the engineered antigen receptor comprises an extracellular binding domain that binds an antigen expressed on a solid cancer cell.

93. The method of claim 85 or claim 86, wherein the engineered antigen receptor comprises an extracellular binding domain that binds an antigen expressed on a liquid cancer cell.

94. The method of claim 85 or claim 86, wherein the engineered antigen receptor comprises an extracellular binding domain that binds an antigen expressed on a malignant B cell.

95. The method of claim 85 or claim 86, wherein the engineered antigen receptor comprises an extracellular binding domain that binds an antigen expressed on a malignant plasma cell.

96. The method of any one of claims 80-95, wherein the retroviral vector is present at an MOI of about 10 to about 30.

97. The method of any one of claims 80-95, wherein the retroviral vector is present at an MOI of about 10 to about 25.

98. The method of any one of claims 80-95, wherein the retroviral vector is present at an MOI of about 10 to about 20.

99. The method of any one of claims 80-95, wherein the retroviral vector is present at an MOI of about 10, about 11, about 12, about 13, about 14, about 15, about 16, about 17, about 18, about 19, about 20, about 21, about 22, about 23, about 24, about 25, about 26, about 27, about 28, about 29 or about 30.

100. The method of any one of claims 80-99, wherein the retroviral vector is a lentiviral vector.

101. The method of any one of claims 80-100, wherein the retroviral vector is derived from a lentivirus selected from the group consisting of: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

102. The method of any one of claims 80-101, wherein the retroviral vector is derived from an HIV lentivirus.

103. The method of any one of claims 80-102, wherein the retroviral vector is derived from an HIV-1 lentivirus.

104. The method of any one of claims 80-103, wherein the population of cells comprises at least about 1×10⁶ T cells to about 1×10⁹ T cells.

105. The method of any one of claims 80-103, wherein the population of cells comprises at least about 1×10⁷ T cells to about 1×10⁹ T cells.

106. The method of any one of claims 80-103, wherein the population of cells comprises at least about 1×10⁸ T cells to about 1×10⁹ T cells.

107. The method of any one of claims 80-103, wherein the population of cells comprises at least about 1×10⁶ T cells to about 1×10⁸ T cells.

108. The method of any one of claims 80-103, wherein the population of cells comprises at least about 1×10⁶ T cells to about 1×10⁷ T cells.

109. The method of any one of claims 80-103, wherein the population of cells comprises at least about 1×10⁶ T cells, at least about 1×10⁷T cells, at least about 1×10⁸ T cells, or at least about 1×10⁹ T cells.

110. The method of any one of claims 80-109, wherein poloxamer 407 is present at a concentration of at least about 100 μg/mL.

111. The method of any one of claims 80-109, wherein poloxamer 407 is present at a concentration of at least about 200 μg/mL.

112. The method of any one of claims 80-109, wherein poloxamer 407 is present at a concentration of at least about 300 μg/mL.

113. The method of any one of claims 80-109, wherein poloxamer 407 is present at a concentration of at least about 400 μg/mL.

114. The method of any one of claims 80-109, wherein poloxamer 407 is present at a concentration of at least about 500 μg/mL.

115. The method of any one of claims 80-114, further comprising a PI3K inhibitor.

116. The method of any one of claims 80-115, further comprising a PI3K inhibitor selected from the group consisting of: BEZ235, LY294002, TG100713, and GDC-0941.

117. The method of any one of claims 80-115, further comprising a PI3K inhibitor selected from the group consisting of: BYL719, GSK2636771, TGX-221, AS25242, CAL-101, and IPI-145.

118. The method of any one of claims 80-115, further comprising the PI3K inhibitor ZSTK474.

119. A method of treating a cancer in a subject in need thereof, comprising administering to the subject a therapeutically effective amount of the composition of any one of claims 1-40 or a therapeutic population of immune effector cells of any one of claims 41-79.

120. The method of claim 119, wherein the cancer is selected from the group consisting of Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer.

121. The method of claim 119, wherein the cancer is pancreatic cancer and the extracellular binding domain binds an epitope of PSCA or MUC1

122. The method of claim 119, wherein the cancer is bladder cancer and the extracellular binding domain binds an epitope of PSCA or MUC1

123. The method of claim 119, wherein the cancer is glioblastoma multiforme and the extracellular binding domain binds an epitope of EPHA2, EGFRvIII, or CSPG4.

124. The method of claim 119, wherein the cancer is lung cancer and the extracellular binding domain binds an epitope of PSCA or GD2.

125. The method of claim 119, wherein the cancer is breast cancer and the extracellular binding domain binds an epitope of CSPG4 or HER2.

126. The method of claim 119, wherein the cancer is melanoma and the extracellular binding domain binds an epitope of CSPG4 or GD2.

127. The method of claim 119, wherein the cancer is a B-cell malignancy and the binding domain binds an epitope of BCMA.

128. A method of treating a hematological malignancy in a subject in need thereof, comprising administering to the subject a therapeutically effective amount of the composition of any one of claims 1-40 or a therapeutic population of immune effector cells of any one of claims 41-79.

129. The method of claim 128, wherein the hematological malignancy is a B-cell malignancy selected from the group consisting of: multiple myeloma (MM), chronic lymphocytic leukemia (CLL), or non-Hodgkin's lymphoma (NHL).

130. The method of claim 128, wherein the MM is selected from the group consisting of: overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

131. The method of claim 128, wherein the NHL is selected from the group consisting of: Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma.


SEQUENCE LISTING (12 sequences)

SEQ ID NO: 1 - protein, 3 aa, Artificial Sequence
  Exemplary linker sequence
GGG

SEQ ID NO: 2 - protein, 5 aa, Artificial Sequence
  Exemplary linker sequence
DGGGS

SEQ ID NO: 3 - protein, 5 aa, Artificial Sequence
  Exemplary linker sequence
TGEKP

SEQ ID NO: 4 - protein, 4 aa, Artificial Sequence
  Exemplary linker sequence
GGRR

SEQ ID NO: 5 - protein, 5 aa, Artificial Sequence
  Exemplary linker sequence
GGGGS

SEQ ID NO: 6 - protein, 14 aa, Artificial Sequence
  Exemplary linker sequence
EGKSSGSGSESKVD

SEQ ID NO: 7 - protein, 18 aa, Artificial Sequence
  Exemplary linker sequence
KESGSVSSEQLAQFRSLD

SEQ ID NO: 8 - protein, 8 aa, Artificial Sequence
  Exemplary linker sequence
GGRRGGGS

SEQ ID NO: 9 - protein, 9 aa, Artificial Sequence
  Exemplary linker sequence
LRQRDGERP

SEQ ID NO: 10 - protein, 12 aa, Artificial Sequence
  Exemplary linker sequence
LRQKDGGGSERP

SEQ ID NO: 11 - protein, 16 aa, Artificial Sequence
  Exemplary linker sequence
LRQKDGGGSGGGSERP

SEQ ID NO: 12 - DNA, 10 bp, Artificial Sequence
  Consensus Kozak sequence
gccrccatgg